(19) United States
(12) Patent Application Publication
(10) Pub. No.: US 2025/0066367 A1
(43) Pub. Date: Feb. 27, 2025

(54) Pyrimidine-Annulated Triazole Derivatives and Their Use in Platelet Aggregation Inhibition

(71) Applicant: BEIJING HONGHUI MEDITECH CO., LTD., Beijing (CN)

(72) Inventors: Pingping ZHANG, Beijing (CN); Xiaoxiong ZHOU, Beijing (CN); Weimin TONG, Beijing (CN); Ye LIU, Beijing (CN)

(21) Appl. No.: 18/719,163

(22) PCT Filed: Dec. 12, 2022
(86) PCT No.: PCT/CN2022/138449

§ 371 Date: Jun. 12, 2024

(30) Foreign Application Priority Data
Dec. 13, 2021 (WO) PCT/CN2021/137533

(51) Int. Cl. C07D 487/04; A61K 31/519; A61K 45/06

(52) U.S. Cl. CPC C07D 487/04; A61K 31/519; A61K 45/06

(57) ABSTRACT

Disclosed relates to novel Pyrimidine annulated derivatives of Formula (I) and the preparation methods of these compounds, and to their use as P2Y₁₂ receptor antagonists in the treatment and/or prevention of thrombosis or related disorders.

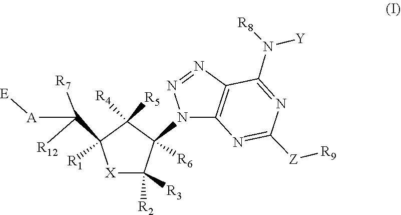

### CROSS REFERENCE TO RELATED APPLICATIONS

This application is a National Stage of International Patent Application No. PCT/CN2022/138449 filed on Dec. 12, 2022, which claims priority to International Patent Application No. PCT/CN2021/137533 filed on Dec. 13, 2021, the contents of which are incorporated by reference in their entirety.

### TECHNICAL FIELD

This disclosure is directed to novel Pyrimidine-annulated triazole derivatives of Formula (I) and the preparation methods of these compounds, and to their use as P2Y₁₂ receptor antagonists in the treatment and/or prevention of thrombosis or related disorders.

### BACKGROUND

Cardiovascular and cerebrovascular diseases are one of the most common causes of mortality and morbidity in the world. Atherothrombosis is an acute complication that develops on the surface of a ruptured atheromotous plague or as a consequence of endothelial erosion which may lead to myocardial infaction or ischemic stroke. (Davi, G et al, New Engl. J. Med., 2007, 357, 2482; Michelson, A. et al, Nat. Rev. Drug Discovery 2010, 9, 154). The blood platelets are the key cellular component in blood for preventing excessive blood loss subsequent to blood vessel injury by rapid formation of a solid blood clot. While the activation and aggregation of platelets are essential for normal hemostasis, under pathological disturbance it is also a key mechanism for triggering acute and potentially life-threatning arterial thrombotic events.

Several converging pathways lead to the platelet aggregation. The final step of the aggregation is the cross-linking of the platelets by binding fibrinogen to the membrane glycoprotein receptor (GP IIb/IIIa). Thrombin can induce platelet aggregation largely independent of other pathways, but substantial quantities of thrombin are unlikely to be present without prior activation of platelets by other mecahnisms. Majority of currently available antiplatelet therapeutics function by either inhibiting platelet agonist production or blocking particular platelet receptors. (Thijs, T, Clin. Chem. Lab. Med., 2010, 48 Suppl.1. S3; Sakhuja. R. et al Curr. Probl. Cardiol. 2010, 35, 123). For example, aspirin reducing the generation of thromboxane A2 (Tohgi, H et al, Stroke, 1992, 23, 1400): abcriximab. epitifibatide and tirofiban etc inhibiting GP IIb/IIIa receptor (Epic Investigators, New Engl. J. Med, 1994, 330, 956; Gibson. C. Am Heart J., 2006, 152, 668; Hartman. G. et al. J Med. Chem. 1992, 35, 4640): clopidogrel. prasugrel. ticlopidine, cangrelor and ticagrelor antagonizing the inducing effect of adenosine-5′-diphosphate (ADP) on purinoceptors (Horiuchi, H, Ann. Med. 2066, 38, 162).

ADP is a key signaling molecule inducing platelet activation and aggregation, thus playing a key role in the initiation and progression of arterial thrombus formation (Gachet, C, Thromb. Haemostasis 2001, 86, 222). The ADP induced platelet aggregation is triggered by binding to two specific P2Y receptors, P2Y₁ and P2Y₁₂. The binding of ADP to those P2Y receptors on the plasma membrane induces the inhibition of adenylyl cyclase and modulates the intracellular signaling pathways like influx and mobilization of intracellular calcium ion, and activation phosphoinositide-3 kinase (PI3K), platelet aggregation and secretion of other mediators, thus amplifying the initial preaggregatory signal (Cateneo, M, Dru News Persp. 2006, 19, 253; Dangelmaier. C. et al. Thromb. Haemost., 2001, 85, 341; Bourdon. D. et al. J. Thromb. Haemost., 2006, 4, 861; Pfefferkom. A. et al. Bioog. Med. Chem. Lett. 2008, 18, 3338). However, while the P2Y₁ is ubiquitously expressed, the P2Y₁₂ receptor is mainly expressed on platelet (Boeynaems, J., et al, Semin. Thromb. Haemast., 2005, 31, 139; Jacobson, K et al. Drug Disc. Today. 2010, 15, 570; ). Besides. experimental studies have shown that sole inhibition of P2Y₁₂ is sufficient to inhibit and prevent platelet aggregation. (Andre, P. et al, J. Cnin. Invest., 2003, 112, 398; Judge. H. et al. Thromb. Haemost., 2010, 103, 1210; Power. R. et al. Expert Rev. Cardiovasc. Ther. 2012, 10, 1261; Ji. X. et al, J. Hematol. Oncol. 2012, 4, 4).

Over the years, enormous efforts have been made to investigate the potential of P2Y₁₂ as a molecular target for treating and preventing pathological atherothrombosis, and a substantial progress has been achieved (Dorsam, R. et al, J Clin. Invest., 2004, 113, 340; Norgard. N. et al. Recent Patents Cardiovasc. Drug Disc. 2008, 3, 194; Patel. P. et al. J. Cardiothor. Vascul. Anesth. 2013, 27, 620); and several P2Y₁₂ inhibition therapeutics have been successfully developed for clinical use (Angiolillo, D., Am. Heart. J., 2008, 156. 510; Michelson, A., Curr. Opin. Hematol. 2009, 16, 371). Such as clopidogrel, prasugrel, ticlopidine, cangrelor and ticagrelor etc. Those P2Y₁₂ inhibitors have been proven to be highly effective in the treatment and prevention of cardiovascular disorders. They are suggested to be used for treating, ameliorating and/or preventing acute myocardial infarction, arterial and venous thrombosis, including primany arterial thrombotic complication of atherosclerosis (such as thrombotic or embolic stroke, transient ischemic attacks or peripheral vascular disease), aterial complications due to intervention in atherosclerotic disease (such as angioplasty and reocclusion following angioplasty), thrombotic complications of surgical or mechanical damages (such as reocclusion following thrombotic therapy), arterial thrombosis, venous thrombosis, conditions in which platelets can contribute to the underlying inflammatory disease processes in the vascular wall (such as atheromatous plague formation/progression, stenosis/restenosis) and angina or unstable angina.

However, it has been observed and documented that all of these clinically available P2Y₁₂ therapeutics have certain undesirable properties or adverse effects related to their respective clinical use. For example, clopidogrel and ticlopidine require to be activated by metabolic enzymes in order to form the active metabolites which in turn irreversibly inhibit the platelet aggregation. They were found to be associated with a high patient individual variation and a significant drug-drug interaction (Collect, J., et al, Lancet, 2009, 373, 309; Simon, T, et al. New Engl. J. Med., 2009, 360, 363; Bhatt, D., N Engl. J Med., 2010, 363, 1909). And the resistance to clopidogrel has been reported (Barragan, P., et al, Catheter Cardiovasc. Interv., 2003, 59, 295; Matetzky, S., Circulation, 2004, 109, 3171). As irreversable inhibitors, prasugrel and clopidogrel have been observed to be associated with enhanced risk of bleeding in the treatment (Koski, R. P & T, 2018, 42, 352; Wiviott, S., N Engl. J Med., 2007, 357, 2001). Cangrelor is only administered parenterally, which limits its potential for a longer-time therapeutic and prophylactic use. Ticagrelor is a potent, reversible inhibitor of platelet aggregation. It has been broadly used as a P2Y₁₂ platelet inhibitor to reduce the thrombotic cardiovascular events in patients with acute coronary syndrome (unstable angina, non-ST elevation myocardial infactin or ST election myocardial infaction) (Husted, S., Cardiovasc. Therap. 2009, 27, 259). Despite its success in clinical use, ticagrelor is found to be associated with serious adverse reactions such as dyspnea, bradycardia etc. Besides, ticagrelor is not recommended for the patients with compromised liver function (Prescribing Information, Brilinta, 2011).

In light of the facts that the cardiovascular and cerebrovascular diseases are still one of the most serious human disease and affecting millions of patients, and that the currently available P2Y₁₂ antiplatelet therapeutics are associated with various undesired properties, there remains a strong need to overcome or at least to mitigate some disadvantages of those P2Y₁₂ inhibitors in clinical use and to provide new, effective and safe pharmaceutical agents to treat and prevent arterial thrombotic events.

Therefore, it is an object of the present disclosure to provide novel compounds, methods and compositions for the treatment and/or prevention of human cardiovascular disorders.

### SUMMARY

The present disclosure includes novel pyrimidine-annulated triazole derivatives, the preparation methods of these compounds and the pharmaceutical compositions comprising such compounds, as well as methods to treat or to prevent arterial thrombosis.

In accordance, the present disclosure provides compounds of the general Formula (I)

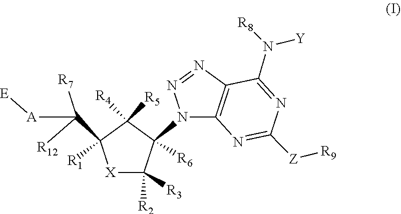

Wherein

X represents O, S, —S(O)— or —S(O)₂—;

Z represents S, O, NH, NR₁₀ or a single bond;

R₁₀ represents C₁₋₆ alkyl;

R₉ represents hydrogen, halogen, C₁₋₈ alkyl, C₂₋₈ alkenyl, C₂₋₈ alkynyl, C₁₋₈ alkyl-carbonyl, C₃₋₈ cycloalkyl, aryl, arakyl, 4-6 membered heterocyclyl or 4-6 membered heterocyclylalkyl, wherein each said group may optionally be substituted by one or more substituents independently selected from halogen, hydroxyl, C₁₋₃ alkoxy, C₁₋₃ alkylthio, amino, N-monoalkylamino, N,N-di-alkylamino, cyano and nitro;

R₈ represents hydrogen, C₁₋₆ alkyl, C₁₋₆alkyloxycarbonyl, N-C₁₋₃ alkylcarbamoyl or N,N-di C₁₋₃ alkylcarbamoyl, wherein said C₁₋₆ alkyl, N-C₁₋₃ alkylcarbamoyl and N,N-di C₁₋₃ alkylcarbamoyl groups may optionally be substituted by one or more substituents selected from hydroxyl, halogen, C₁₋₃ alkoxy;

Y represents H, C₁₋₈ alkyl, C₁₋₆ alkoxy-C₁₋₆ alkyl, C₁₋₆ alkyl-thio-C₁₋₆ alkyl, C₃₋₈ cycloalkyl, C₃₋₈ cycloalkyl-C₁₋₃ alkyl, phenyl-C₁₋₃ alkyl, phenyl-C₃₋₈ cycloalkyl, 4-10 membered heterocyclyl, 4-10 membered heterocyclyl-C₁₋₃ alkyl or 4-10 membered heterocyclyl-C₃₋₈ cycloalkyl, wherein each said group may optionally be substituted by one or more substituents independently selected from halogen, hydroxyl, cyano, nitro, C₁₋₃ alkyl, C₂₋₆ alkenyl, C₂₋₆ alkynyl, C₁₋₃ alkoxy, C36 cycloalkyl, C₁₋₃ alkoxy-C₁₋₃ alkyl, halo-C₁₋₃ alkyl, amino, N-alkylamino, and N,N-dialkylamino;

R₁ represents H, cyano, C₁₋₃ alkyl, halo-C₁₋₃ alkyl, hydroxylmethyl, 2-hydroxyethyl, C₁₋₈ alkylcarboxylmethyl, or benzoylmethyl, wherein the said C₁₋₈ alkylcarboxylmethyl, and benzoylmethyl groups may optionally be substituted by one or more substituents selected from halogen, carboxyl and C₁₋₆ alkoxy-carbonyl;

R₂ and R₃ independently represent hydrogen, C₁₋₃ alkyl, halo-C₁₋₃ alkyl, C₁₋₃alkoxy, hydroxylmethyl, 2-hydroxyethyl, 3-hydroxypropyl, —C(O)OH, —CH₂C(O)OH, C₁₋₆ alkoxycarbonyl, C₁₋₆ alkoxycarbonylmethyl or cyano;

Or R₂ and R₃ together with the carbon to which they are bonded form a C₃₋₅ carbocyclyl, wherein the said carbocyclyl may optionally be substituted by one or more substituents selected from halogen, OH, C₁₋₃ alkyl or C₁₋₃ alkoxy:

R₄ and R₅ independently represent hydrogen, halogen, hydroxyl, C₁₋₃ alkyl, halo-C₁₋₃ alkyl, C₁₋₃ alkoxy, hydroxylmethyl, 2-hydroxyethyl, 3-hydroxypropyl, —C(O)OH, C₁₋₆ alkoxycarbonyl, CI-6 alkoxycarbonylmethyl, cyano, amino, N-alkylamino or N, N-dialkylamino;

Or R₄ and R₅ together with the carbon to which they are bonded form a C₃₋₅ carbocyclyl, wherein the said carbocyclyl may optionally be substituted by one or more substituents with halogen, OH, C₁₋₃ alkyl or C₁₋₃ alkoxy:

R₆ represents hydrogen, cyano, C₁₋₃ alkyl, hydroxylmethyl or 2-hydroxyethyl;

R₇ represents hydrogen, F, C₁₋₃ alkyl, hydroxymethyl, 2-hydroxyethyl or carboxymethyl; or R₇ and R₁₂ together represent an oxo;

A represents a single bond, hydrogen, O, S, —CH₂—, —CH₂CH₂—, CHF, CF₂ or —C(O)—;

E represents hydrogen, F, OH, R₁₁O—, R₁₁OC(O)—, R₁₁C(O)—, R₁₁C(O)O— or a substructure represented by G1, G2 or G3;

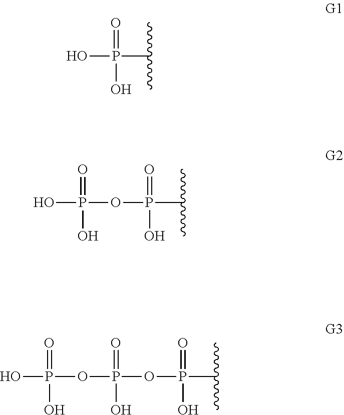 [SMILES: *P(=O)(O)O ; *P(=O)(O)OP(=O)(O)O ; *P(=O)(O)OP(=O)(O)OP(=O)(O)O]

R₁₁ represents hydrogen, C₁₋₈ alkyl, C₃₋₈ alkenyl, C₃₋₈ alkynyl, C₃₋₈ cycloalkyl, benzyl, aryl and heterocyclyl, wherein the said groups may optionally be substituted by one or more substituents selected from halogen, OH, nitro, cyano, C₁₋₃ alkyl, C₁₋₃ alkoxy, carboxyl, C₁₋₆ alkoxycarbonyl and C₁₋₆ alkoxycarbonyloxy;

R₁₂ represents hydrogen; or R₁₂ and R₇ together represent an oxo;

With the proviso that Y is not H when R₈ is H;

And with the proviso that R₈ is not H when Y is H;

And with the proviso that R₉ is not H when Z is a single bond;

Or a pharmaceutically acceptable salt or prodrug thereof.

In one embodiment of the present disclosure, there are provided compounds of the general Formula (I) wherein X is O and R₆ is H.

With the proviso that Y is not H when R₈ is H;

And with the proviso that R₈ is not H when Y is H;

And with the proviso that R₉ is not H when Z is a single bond;

Or a pharmaceutically acceptable salt or prodrug thereof.

In another embodiment of the present disclosure, there are provided compounds of the general Formula (I) wherein X is O; R₁ is H; R₆ is H; R₁₂ is H; R₈ is H and Z is S.

In still another embodiment of the present disclosure, there are provided compounds of the general Formula (I) wherein X is O; R₁ is H; R₆ is H; R₁₂ is H; R₈ is H; Z is S; R is H; R₉ is C₁₋₅ alkyl, C₃₋₈ cycloalkyl or a phenyl group; Y is a cyclopropyl that is substituted with a phenyl or heterocyclyl wherein the said phenyl or heterocyclyl may optionally be substituted by one or more substituents independently selected from halogen, CN, nitro, OH, C₁₋₃ alkyl, C₁₋₃alkoxy, C₃₋₆cycloalkyl, amino, N-alkylamino and N,N-dialkylamino.

In still another embodiment of the present disclosure, there are provided compounds of the general Formula (I) wherein A is a single bond, hydrogen or O; X is O; R₁ is H; R₆ is H; R₁₂ is H; R₈ is H; Z is S; R₇ is H; R₉ is C₁₋₅ alkyl, C₃₋₈ cycloalkyl or a phenyl group; Y is a cyclopropyl that is substituted with a phenyl or heterocyclyl wherein the said phenyl or heterocyclyl may optionally be substituted by one or more substituents independently selected from halogen, CN, nitro, OH, C₁₋₃ alkyl, C₁₋₃ alkoxy, C₃₋₆ cycloalkyl, amino, N-alkylamino and N,N-dialkylamino.

In still another embodiment of the present disclosure, there are provided compounds of the general Formula (I) wherein R₁ is H; R₆ is H; R₁₂ is H; R₈ is H; Z is S; R₄ and R₅ are independently selected from H, OH, fluorine and hydroxymethyl.

In still another embodiment of the present disclosure there are provide compounds of general Formula (I) wherein R₁ is H; R₆ is H; R₁₂is H; R₈ is H; Z is S; R₃ is H; A is O or a single bond; E is H, R₁₁O— or R₁₁C(O)—; and R₁₁ is C₁₋₈ alkyl, benzyl or phenyl, wherein the said benzyl, phenyl groups may optionally be substituted by one or more substituents selected from halogen, OH, C₁₋₃ alkyl, C₁₋₃ alkoxy, carboxyl, C₁₋₆ alkoxycarbonyl and C₁₋₆ alkoxycarbonyloxy.

In still another embodiment of the present disclosure there are provided compounds of general Formula (I) wherein R₁ is H; R₆ is H; R₁₂ is H; R₈ is H; Z is S; R₃ is H; and R₂ is hydroxymethyl.

In still another embodiment of the present disclosure there are provided compounds of general Formula (I) wherein Y is a cyclopropyl that is substituted with phenyl, wherein the said phenyl is substituted by 1 to 4 fluorine atoms.

In still another embodiment of the present disclosure, there are provided compounds of general Formula (I), selected from:
((2R,3R,4R,5S)-3-(7-(((1R,2S)-2-(3,4-difluorophenyl)cyclopropyl)amino)-5-(propylthio)-3H-[1,2,3]triazolo[4,5-d]pyrimidin-3-yl)- 4-hydroxytetrahydrofuran-2,5-diyl)dimethanol;
((2R,3R,5R)-3-(7-(((1R,2S)-2-(3,4-difluorophenyl)cyclopropyl)amino)-5-(propylthio)-3H-[1,2,3]triazolo[4,5-d]pyrimidin-3- yl)tetrahydrofuran-2,5-diyl)dimethanol;
(2S,3R,4S)-4-(7-(((1R,2S)-2-(3,4-difluorophenyl)cyclopropyl)amino)-5-(propylthio)-3H-[1,2,3]triazolo[4,5-d]pyrimidin-3-yl)-2- (hydroxymethyl)tetrahydrofuran-3-ol;
((2R,4R)-4-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2-yl)methanol;
((2R,3S,4S,5S)-3-(7-(((1R,2S)-2-(3,4-difluorophenyl)cyclopropyl)amino)-5-(propylthio)-3H-[1,2,3]triazolo[4,5-d]pyrimidin-3-yl)- 4-fluorotetrahydrofuran-2,5-diyl)dimethanol;
((2R,3S,4R,5S)-3-(7-(((1R,2S)-2-(3,4-difluorophenyl)cyclopropyl)amino)-5-(propylthio)-3H-[1,2,3]triazolo[4,5-d]pyrimidin-3-yl)- 4-fluorotetrahydrofuran-2,5-diyl)dimethanol;
(2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5- (hydroxymethyl)tetrahydrofuran-2-carboxylic acid;
(2R,4R,5R)-4-(7-(((1R,2S)-2-(3,4-difluorophenyl)cyclopropyl)amino)-5-(propylsulfinyl)-3H-[1,2,3]triazolo[4,5-d]pyrimidin-3-yl)-5-(hydroxymethyl)tetrahydrofuran-2-carboxylic acid;
((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5- (methoxymethyl)tetrahydrofuran-2-yl) methanol;
2-((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl) ethan-1-ol;
2-(((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl)methoxy)ethan-1-ol;
3-((2S,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl)propanoic acid;
2-((((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3- yl)-5-(hydroxymethyl)tetrahydrofuran-2-yl)methoxy)methyl)benzoic acid;
2-(((2R,4R,5R)-4-(7-(((1S,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl)methoxy)acetic acid;
or a pharmaceutically acceptable salt or prodrug thereof.

In still another embodiment of the present disclosure there is provided a pharmaceutical composition for the treatment, amelioration or prevention of platelet-mediated thrombotic diseases, disorders or conditions including, but not limited to, myocardial infarction, thrombotic stroke, transient ischemic attacks, peripheral vascular disease, angina, and unstable angina in a host comprising an effective amount of a compound of the general Formula (I).

In still another embodiment of the present disclosure there is provided a pharmaceutical composition as described above, which further comprises one or more additional antiplatelet, anticoagulant, antifibrinolytic agents or other therapeutic agents having cardiovascular effects. Such additional agents may include the following non-limiting examples: aspiring, triflusal, heparins, cilostazol, vorapaxar, abciximab, epitifibatide, tirofiban, dipyridamole, thromboxane synthase inhibitor, terutroban, apixaban, rivaroxaban dabigatran, apixaban, huridin, edoxaban, inogatran, ximelagatran and vitamin K antagonists.

In still another embodiment of the disclosure there is provided a compound of the general Formula (I) for use in therapy.

In still another embodiment of the present disclosure there is provided a compound of the general Formula (I), for use in the treatment amelioration or prevention of a platelet-mediated thrombotic diseases, disorders or conditions including, but not limited to, myocardial infarction, thrombotic stroke, transient ischaemic attacks, peripheral vascular disease, angina, and unstable angina.

In still another embodiment of the present disclosure there is provided a compound of the general Formula (I), for use in the treatments, amelioration or preventions as described above, which further comprises one or more additional therapeutic agents selected from the group consisting of antiplatelet, anticoagulant and anti-fibrinolytic agents or any combination thereof. Such additional agents may include the following non-limiting examples: aspirin, triflusal, heparins, cilostazol, vorapaxar, abciximab, epitifibatide, tirofiban, dipyridamole, thromboxane synthase inhibitor, terutroban, apixaban, rivaroxaban dabigatran, huridin, edoxaban, inogatran, ximelagatran and vitamin K antagonists.

In still another embodiment of the present disclosure there is provided use of a compound of the general Formula (I), in the manufacture of a medicament for treatment, amelioration or prevention of platelet-mediated thrombotic diseases, disorders or conditions including, but not limited to, myocardial infarction, thrombotic stroke, transient ischaemic attacks, peripheral vascular disease, angina, and unstable angina.

In still another embodiment aspect of the present disclosure there is provided use of a compound of the general Formula (I), in the manufacture of a medicament for treatments or preventions as described above, which further comprises one or more additional therapeutic agents selected from the group consisting of antiplatelet, anticoagulant and anti-fibrinolytic agents, or any combination thereof. Such additional agents may include the following non-limiting examples: aspirin, triflusal, heparins, cilostazol, vorapaxar, abciximab, epitifibatide, tirofiban, dipyridamole, thromboxane synthase inhibitor, terutroban, apixaban, rivaroxaban dabigatran, huridin, edoxaban, inogatran, ximelagatran and vitamin K antagonists.

In still another embodiment of the present disclosure there is provided a method for the treatment amelioration or prevention of a platelet-mediated thrombotic diseases, disorders or conditions including, but not limited to, myocardial infarction, thrombotic stroke, transient ischaemic attacks, peripheral vascular disease, angina, and unstable angina in a subject in need thereof, comprising administering a therapeutically effective amount of a compound of the general Formula (I).

In another aspect of the disclosure there is provided a method as described above, which further comprises one or more additional therapeutic agents selected from the group consisting of antiplatelet, anticoagulant and anti-fibrinolytic agents, or any combination thereof. Such additional agents may include the following non-limiting examples: aspirin, triflusal, heparins, cilostazol, vorapaxar, abciximab, epitifibatide, tirofiban, dipyridamole, thromboxane synthase inhibitor, terutroban, apixaban, rivaroxaban dabigatran, huridin, edoxaban, inogatran, ximelagatran and vitamin K antagonists.

The individual components of such combinations may be administered either sequentially or simultaneously in separate or combined pharmaceutical formulations.

### DETAILED DESCRIPTION OF THE EMBODIMENTS

### Definitions

Whenever used foregoing and hereinafter, the term “compounds of Formula (I)”, or “the compounds of the disclosure”, or “the compounds of the present disclosure” or similar terms, it is meant to include the compounds of Formula (I), their pharmaceutically acceptable prodrugs, salts, solvates, quaternary amines and metal complexes.

The term “prodrug” as used throughout this text means the pharmacologically acceptable derivatives such as esters, carbamate, carbonate, ether, amides, phosphate esters, phosphonate esters, phosphoramidate, phosphonamidate, phosphodiamide, phosphonodiamide. The said derivatives are made such that the resulting in vivo biotransformation product of the derivative is the active drug as defined in the compounds of Formula (I). The references describing prodrugs generally are hereby incorporated (Goodman and Gilman, The Pharmacological Basis of Therapeutics, 8^{th} ed, McGraw-Hill, Int. Ed. 1992, “Biotransformation of Drugs”, p 13-15; H. Bundgaard, Design of Prodrugs, H. Bundgaard ed. Elsevier Science Publisher, 1985; M. Taylor, Adv. Drug Delivery 1996, 19, 131; H. Bundgaard, Drugs of the Future, 1991, 16, 443; A. Simplicio, Molecules, 2008, 13, 519; P. Ettmayer, J. Med. Chem. 2004, 47, 2393). Particularly relevant are the prodrugs described for making nucleoside or nucleotide prodrugs (S. Hecker et al, J. Med. Chem., 2008, 51, 2328; P. Poijarvi-Virta et al, Current Med. Chem. 2006, 13, 3441; N. Gisch et al, J Med. Chem. 2008, 51, 6752; L. Wiebe et al. Adv. Drug Delivery Rev. 1999, 39, 63; J. Cooperwood et al, Nucleoside and Nucleotide Prodrugs, in Recent Advances in Nucleosides: Chemistry and Chemotherapy, C.K.Chu ed. Elsevier, 2002, p. 91-147), Prodrugs preferably have excellent aqueous solubility, increased bioavailability and are readily metabolized into the compounds of Formula (I) in vivo. The prodrugs which can increase the cell membrane permeation of the compounds of the disclosure are particularly preferred and are a part of this disclosure. Prodrugs of a compound of the present disclosure may be prepared by modifying functional groups present in the compounds in such a way that the modifications are cleaved, either by routine manipulation or in vivo, to the parent compound.

Preferred are pharmaceutically acceptable ester, ether, carbonate, phosphodiester, phosphonomonoester, phosphotriester, phophonodiester, phosphoramidate, phosphonamidate, phosphodiamide, phosphonodiamide and carbamate prodrugs that are hydrolysable in vivo and are derived from those compounds of Formula (I) having a hydroxy and/or an amino and/or a phosphate and/or a phosphonate group. An in vivo hydrolysable ester, ether, carbonate, phosphodiester, phosphonomonoester, phosphotriester, phophonodiester, phosphoramidate phosphonamidate, phosphodiamide, phosphonodiamide and carbamate are hydrolysed in the human or animal body to produce the parent alcohol, amine, phosphate or phosphonate which are present in the compounds of the disclosure. The hydrolysis of the prodrugs can be of chemical hydrolysis or enzymatic hydrolysis, with the enzymatic hydrolysis as the preferred mechanism. The enzymes commonly involved in the nucleoside/nucleotide prodrug hydrolysis include, but not limited to, carboxyesterases, cholinesterases, cathepsin A, lipase, valacyclovirase, paraoxonases.

There is also provided pharmaceutically acceptable salts of the compounds of Formula (I) of the present disclosure. By the term “a pharmaceutically acceptable salt” is meant those derived from pharmaceutically acceptable inorganic and organic acids and bases. A suitable pharmaceutically acceptable salt of a compound of the disclosure is, for example, an acid-addition salt of a compound of the disclosure which is sufficiently basic, for example, an acid-addition salt with, for example, an inorganic or organic acid, for example hydrochloric, hydrobromic, nitric, methansulphonic, sulphuric, phosphoric, trifluoroacetic, para-toluene sulphonic, 2-mesitylen sulphonic, citric, acetic, tartaric, fumaric, lactic, succinic, malic, malonic, maleic, 1,2-ethanedisulphonic, adipic, aspartic, benzenesulphonic, benzoic, ethanesulphonic or nicotinic acid. In addition a suitable pharmaceutically acceptable salt of a compound of the disclosure, is, for example, a base-addition salt of a compound of the disclosure which is sufficiently acidic, for example, a metal salt, for example, sodium, potassium, calcium, magnesium, zinc or aluminum, an ammonium salt, a salt with an organic base which affords a physiologically acceptable cation, which includes quarternary ammonium ion, for example methylamine, ethylamine, diethylamine, trimethylamine, tert-butylamine, triethylamine, dibenzylamine, N,N-dibenzylethylamine, cyclohexylethylamine, tris-(2-hydroxyethyl)amine, hydroxyethyl diethylamine, (IR,2S)-2-hydroxyinden-l-amine, morpholine, N-methylpiperidine, N-ethylpiperidine, piperazine, methylpiperazine, adamantylamine, choline hydroxide, tetrabutylammonium hydroxide, tris-(hydroxymethyl)methylamine hydroxide, L-arginine, N-methyl D-glucamine, lysine, arginine and the like.

Certain compounds of the present disclosure may exist as solvates or hydrates. It is to be understood that the present disclosure encompasses all such solvates or hydrates.

The compounds of the present disclosure may also contain unnatural proportions of atomic isotopes at one or more of the atoms that constitute such compounds. For example, the compounds may be radiolabeled with radioactive isotopes, such as for example tritium (³H), deuterium (²H), iodine-125 (¹²⁵I) or carbon-14 (¹⁴C). All isotopic variations of the compounds of the present disclosure, whether radioactive or not, are intended to be encompassed within the scope of the present disclosure.

Where tautomers exist in the compounds of the disclosure, we disclose all individual tautomeric forms and combinations of these as individual specific embodiments of the disclosure.

The compounds according to the disclosure have the core structures of a 5-membered tetrahydrofuran, tetrahydrothiophene, tetrahydrothiophene-1-oxide or tetrahydrothiophene-1,1-dioxide ring with defined stereochemistry at 2-, 3-, 4- and 5-positions of the said 5-membered rings. The compounds of Formula (I) may contain one or more asymmetrically substituted carbon atoms, asymmetric or chiral centres. The presence of one or more of these asymmetric centres in the compounds according to the disclosure can give rise to stereochemically isomeric forms, stereoisomers (e.g. racemate, enantiomer, diastereomer or E- or Z-isomer). Unless the stereochemistry is clearly defined by the chemical structures, in each case the disclosure is to be understood to possibly extend to all such stereoisomers, both in pure form and mixed with each other, including enantiomers and diastereomers, and mixtures including racemic mixtures thereof.

It will be appreciated that the compounds of Formula (I) may have metal binding, chelating or complex forming properties and therefore may exist as metal complexes or metal chelates. Such metalated derivatives of the compounds of Formula (I) are intended to be included within the scope of the disclosure.

The scientific and technological terms and nomenclatures used foregoing and hereinafter have the same meaning as commonly understood by a person of ordinary skill in the art; in addition, the following definitions shall apply throughout the specification and the appended claims unless specifically stated otherwise:

The term “halogen” denotes fluoro, chloro, bromo and iodo groups.

In this document, the sign “-” is sometimes added to clarify which bond serves as a connection point. For example, heterocyclyl-C₁₋₃alkyl represents a C₁₋₃alkyl radical substituted with a heterocyclyl moiety. Further, RO— represents a radical wherein R is bonded to an oxygen atom and the said oxygen atom is at the connecting point for the whole radical.

The term “C₁₋ₙalkyl” denotes a straight or branched saturated alkyl group having 1 to n carbon atoms, wherein “n” is an integer from 1 to 18. Examples of “n” include 2, 3, 4, 5, 6, 7, 8 and 18.Examples of said alkyl include, but are not limited to, methyl, ethyl, propyl isopropyl, butyl, isobutyl, tert-butyl, pentyl, isopentyl and hexyl.

The term “C₂₋ₙalkenyl” denotes a straight or branched alkenyl group having saturated carbon-carbon bonds and at least one carbon-carbon double bond, and having 2 to n carbon atoms, wherein “n” is an integer from 2 to 18. Examples of “n” include 2, 3, 4, 5, 6, 7, 8 and 18. Examples of said alkenyl include, but are not limited to, ethenyl, 1-propenyl, 2-propenyl, isopropenyl and butenyl.

The term “C₃₋₆cycloalkyl” denotes a saturated monocyclic ring having 3 to 6 carbon atoms. Examples of said cycloalkyl include, but are not limited to cyclopropyl, cyclobutyl, cyclopentyl and cyclohexyl.

The term “C₁₋ₙalkoxy” denotes a C₁₋ₙalkyl as defined above linked to oxygen, i.e. C₁₋ₙalkyl-O—. Examples of said alkoxy include, but are not limited to, methoxy, ethoxy, n-propoxy, isopropoxy and butyloxy.

The term “aryl” denotes an aromatic ring or an aromatic ring fused with aromatic or non-aromatic carbocyclic or heterocyclic ring or rings forming a mono-, bi- or tricyclic ring system composed of 6-14 carbon atoms, preferably 6-10 carbon atoms. Examples of said aryl include, but are not limited to, phenyl, naphthyl, biphenyl, 2-naphthanyl, tetrahydronaphthyl, 2-indenyl, 4-indenyl and indanyl.

The term “aryl-C₁₋ₙalkyl” denotes a C₁₋ₙalkyl as defined above substituted with an aryl as defined above.

The term “aryl-C₂₋ₙalkenyl” denotes a C₂₋ₙalkenyl as defined above substituted with an aryl as defined above.

The term “heterocyclyl” denotes a saturated, partially unsaturated or aromatic mono-, bi- or tricyclic ring system composed of 4-18 atoms in which 1, 2, 3 or 4 of the atoms in the ring(s) is an element other than carbon independently selected from one or more of nitrogen, oxygen or sulphur. Examples of said heterocyclyl include, but are not limited to pyrrolidino, piperidino, oxetanyl, pyridinyl, piperazino, morpholino, dioxanyl, thiomorpholino, furanyl, tetrahydrofuranyl, pyranyl, tetrahydropyranyl, thiazolyl, oxazolyl, thiazinolyl, imidazolyl, triazolyl, tetrazolyl, isoxazolyl, oxadiazolyl, indolinyl, isoindolinyl, isoquinolinyl, quinazolinyl, benzimidazolyl, benzoxazolyl, benzisoxazolyl, indolyl and isoindolinyl.

The term “C₃₋ₚcycloalkyl-C₁₋ₙalkyl” denotes a C₁₋ₙalkyl as defined above substituted with a C₃₋ₚcycloalkyl as defined above.

Unless otherwise indicated, the alkyl, alkenyl, alkoxy and cycloalkyl, (such as in C₁-C₆alkyl, C₂-C₆alkenyl, C₃-C₆cycloalkyl and the like) are independently optionally substituted with one or more substituents independently selected from: halogen, hydroxyl, amino, oxo, mercapto, amido, cyano, azido, nitro, C₁-C₃alkyl, C₂-C₄alkenyl, C₂-C₄alkynyl, C₃-C₆cycloalkyl, C₁-C₄alkoxy. It should be noted that the radical positions on any molecular moiety used in the definitions may be anywhere on such a moiety as long as it is chemically permitted and stable.

The term “optionally substituted” as used herein, means that substitution is optional, i.e. there may or may not be substitution. For instance, the expression “alkyl group optionally substituted with one or more substituents” means that the alkyl group is substituted by zero, one or more substituents.

The term “substituted” refers to a molecule wherein at least one hydrogen atom is replaced with a substituent.

Radicals used in the definitions of the variables include all possible isomers unless otherwise indicated. For instance, pyridyl includes 2-pyridyl, 3-pyridyl and 4-pyridyl; pentyl includes 1-pentyl, 2-pentyl, 3-pentyl and the like. When any variable occurs more than one time in any constituent, each definition is independent.

The term “subject” represents any mammals including humans. In one embodiment of the disclosure, the subject is human.

The term “independently” is used herein to indicate that the variable, which is independently applied, varies independently from application to application.

It is understood that some compounds of Formula (I) may be further modified to obtain desired compounds which can also be represented by Formula (I). The methods for such modifications depend on the structures of the desired products and the structures of the compounds of Formula (I) as the starting materials. Such modification reaction may involve deprotection, substitution, addition, oxidation, reduction and other chemical transformations which are common in organic syntheses.

### Pharmaceutical Preparation

It will be appreciated that the amount of a compound of Formula (I) of the present disclosure required for use in treatment will vary not only with the particular compound selected but also with the route of administration, the nature of the condition for which treatment is required and the age, weight and condition of the patient and will be ultimately at the discretion of the attendant physician. In general, however a suitable dose may be in the range of from about 0.005 to about 30 mg/kg of body weight per day, preferably in the range of 0.05 to 20 mg/kg/day.

The desired dose is conveniently presented in a single dose or as divided dose administered at appropriate intervals, for example as two, three, four or more doses per day. Dependent on the need of the treatment and/or prevention, the desired dose may also be, for example, once every two days, once every three days, or even once a week.

The compound is conveniently administered in unit dosage form: for example. containing 0.5 to 1500 mg, conveniently 1 to 1000 mg, most conveniently 5 to 700 mg of active ingredient per unit dosage form.

The compounds of the disclosure will normally be administrated via the oral, parenteral, intravenous, intramuscular, subcutaneous or other injectable ways, buccal, rectal, vaginal, transdermal and/or nasal route and/or via inhalation, in the form of pharmaceutical preparations comprising the active ingredient or a pharmaceutically acceptable salt or prodrug or solvate thereof, or a solvate of such a salt, in a pharmaceutically acceptable dosage form. Depending upon the disorder and patient to be treated and the route of administration, the compositions may be administered at varying doses.

While it is possible that, for use in therapy, a compound of Formula (I) of the present disclosure may be administered as the raw chemical, it is preferable according to one embodiment of the disclosure, to present the active ingredient as a pharmaceutical composition. The disclosure thus further provides a pharmaceutical composition comprising a compound of Formula (I) or a pharmaceutically acceptable salt or prodrug thereof together with one or more pharmaceutically acceptable carriers. The carrier(s) must be “acceptable” in the sense of being compatible with the other ingredients of the formulation and not deleterious to the recipient thereof. According to one embodiment of the present disclosure, pharmaceutical formulations include but are not limited to those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods according to this embodiment include the step of bringing into association the active compound with liquid carriers or finely divided solid carriers or both and then, if necessary, shaping the product into the desired composition.

Pharmaceutical compositions suitable for oral administration are conveniently presented as discrete units such as capsules, cachets or tablets each containing a predetermined amount of the active ingredient; as a powder or granules. In another embodiment, the formulation is presented as a solution, a suspension or as an emulsion. The active ingredient is alternatively presented as a bolus, electuary or paste.

Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds of Formula (I) may be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

The above described formulations are adapted to give sustained release of the active ingredient.

### General Synthetic Methods

The present disclosure also relates to methods of making the compounds of the disclosure. The compounds may be prepared by any of the applicable methods and techniques of organic synthesis. Many such methods and techniques are well known in the art and some of the known methods techniques are elaborated in Compendium of Organic Synthetic Methods, in 12 volumes (John Wiley & Sons, New York); Advanced Organic Chemistry, 5 ed. M. Smith & J. March (John Wiley & Sons, New York. 2001); Comprehensive Organic Synthesis. Selectivity. Strategy & Efficiency in Modern Organic Chemistry, in 9 Volumes. Barry M. Trost, Editor-in-Chief (Pergamon Press, New York, 1993) and Chemistry of Nucleosides and Nucleotides, Townsend. L. B., Ed. (Plenum Press; New York, 1988).

A number of exemplary methods for the preparation of the compounds of the disclosure are provided below. These methods are intended to illustrate the nature of such preparations and are not intended to limit the scope of applicable methods. Alternative routes, which will be readily apparent to the ordinary skilled organic chemist, may alternatively be used to synthesize the compounds of the disclosure or their intermediates as illustrated by the general schemes and the preparative examples below. The preparation of the various starting materials used in the schemes is well within the skill of persons versed in the art.

In the course of the process described below for the preparation of compounds of Formula (I), functional groups in starting materials which are prone to participate in undesired side reactions, especially amino, amide, carboxy, hydroxy, phosphate, phosphonate and mercapto groups, may be protected by suitable conventional protecting groups which are customarily used in the organic synthesis. Those protecting groups may already be present in the precursors and they are intended to protect the functional groups in question against undesired secondary reactions, such as acylation, etherification, esterification, alkylation, oxidation, reduction, solvolysis, etc. In certain cases, the protecting groups can additionally cause the reactions to proceed selectively, for example regioselectively or stereoselectively. It is characteristic of protecting groups that they can be removed easily, i.e. without undesired secondary reactions taking place, for example by acid treatment, fluoride treatment, solvolysis, reduction, or by photolysis. The protection of functional groups by such protecting groups, the protecting groups themselves, and the reactions for their removal are described, for example, in standard works such as M. Schelhaas et al, Angew. Chem, Internatl. Ed. (1996), 35 (18), 2056 and GDT.W. Greene and P. G. M. Wuts “Protective Groups in Organic Synthesis” John Wiley & Sons, Inc., New York 1999.

Schemes 1-6 illustrate different processes for synthesizing compounds of Formula (I) or a compound which may be converted to a compound of Formula (I).

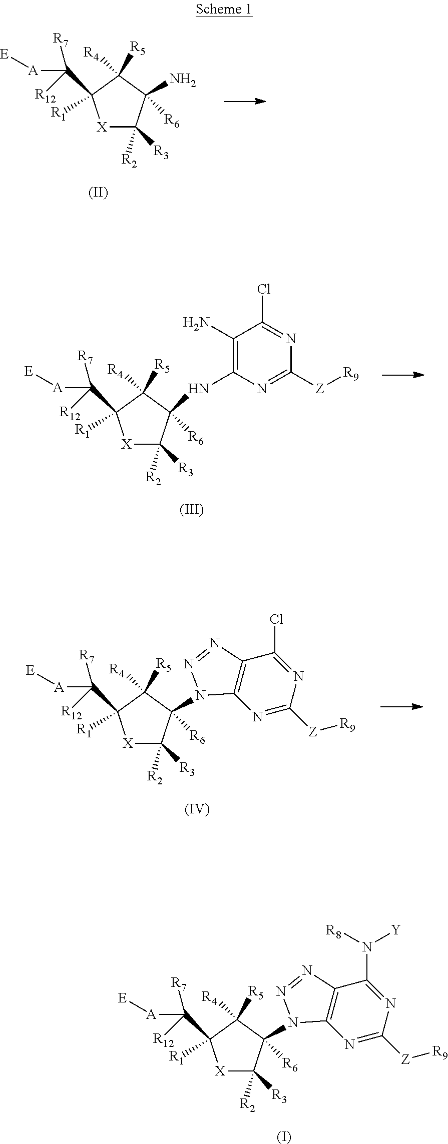

Scheme 1 describes a general method for the preparation of compounds according to Formula (I). The appropriate starting material, the five-membered heterocyles of Formula (II) wherein R₁, R₂, R₃, R₄, R₅, R₆, R₇, R₁₂, X, A and E are as defined for Formula (I), can be prepared using the applicable methods and techniques in organic synthesis well known in literature and to the organic chemists versed in the art (Smith M., et al. Compendium of Organic Synthetic Methods, in 12 volumes (John Wiley & Sons, New York; Comprehensive Organic Synthesis. Selectivity. Strategy & Efficiency in Modern Organic Chemistry, in 9 Volumes. Barry M. Trost, Editor-in-Chief (Pergamon Press, New York, 1993). The compounds of Formula (II) are optionally properly-protected wherever necessary. They are reacted with an appropriately substituted 4,6-dichloro-pyrimidine-5-amine in a polar solvent in the presence of a base to provide the compound of Formula (III). The suitable base for the reaction includes triethylamine, DIPEA, DMAP, potassium carbonate, sodium carbonate, cesium carbonate, DBU, pyridines, or other organic or inorganic bases common for such reactions. The solvents for the reaction include DMF, THE, 1,4-dioxane, acetonitrile, ethanol and other common solvents suitable for substitution reaction (Springthorpe, B. et al, Bioorg. Med. Chem. Lett., 2007, 17, 6013; Zhang. H., et al, Bioorg. Med. Chem. Lett., 2012, 22, 3598). Alternatively, the compound of (II) can react with an appropriately substituted 4,6-dichloro-5-nitro-pyrimidine using a similar method, the obtained intermediate is subsequently converted to the compound of Formula (III) through the reduction of the nitro group into an amino group (Tu, W. et al, Bioorg. Med. Chem. Lett., 2014, 24, 141). The reduction conditions include Fe in AcOH, Fe/FeSO₄ in aqueous solvents, Zn in aprotic solvents, catalytic hydrogenation and metal hydride, etc. The compounds of Formula (III) is converted to triazolopyrimidine of Formula (IV) under diazotization conditions such as isoamylnitrile in acetonitrile, NaNO₂ in AcOH/H₂O, etc. The compounds of Formula (IV) are reacted on their pyrimidine ring by the substitution of an appropriate amine in an organic solvent such as MeCN, DMF, THE, dichloromethane, dichloroethane, dioxane or a mixture of solvents. An extra base is preferred in the reaction, such as triethylamine, dipropylethylamine, 2,6-lutidine, pyridine, DMAP, DBU, etc. The resulting intermediates can be optionally further modified to give desired compounds of Formula (I) after the deprotection.

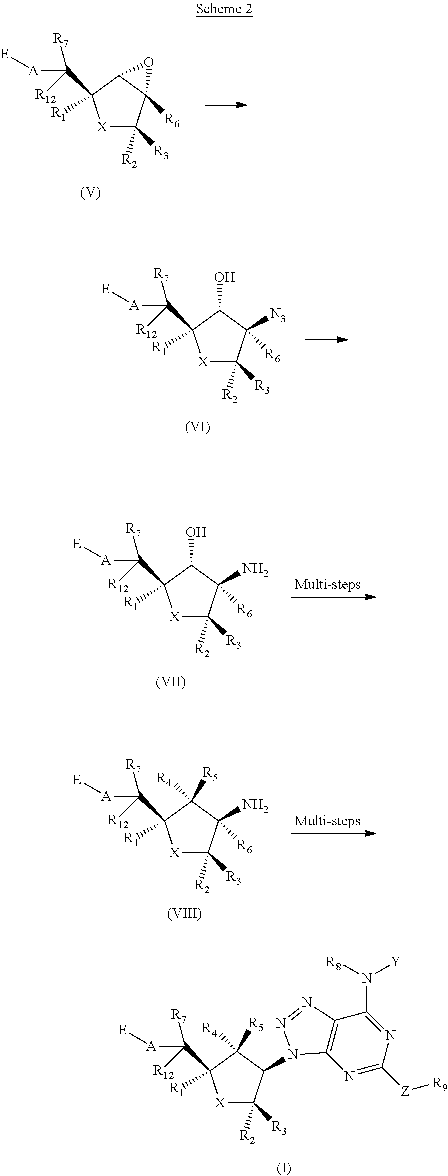

Scheme 2 describes another method for the preparation of compounds according to Formula (I). The appropriate five-membered heterocylic epoxide, Formula (V), wherein R₁, R₂, R₃, R₆, R₇, R₁₂, X, A and E are as defined for Formula (I) and are optionally properly-protected wherever necessary, are reacted with an azide, such as potassium azide, sodium azide, tretrabutylammoniumazide and so on, in an suitable solvent or a mixture of solvents. The azido group of the resulting compound of Formula (VI) can be reduced to amine, using appropriate reaction conditions such as metal hydrides, palladium catalyzed hydrogenation, triphenylphosphine and a method known in literature (Scriven E et al, Chem Rev. 1988, 88, 297; Rolla, F. J Org. Che. 198, 47, 4327; Kim, B, et al, Tetrahedron Lett. 1998, 39, 6921; Staudinger, H.; Helv. Chim. Acta, 1919, 2, 635). The compound of Formula (VI), after appropriate protection of the amino group, can be converted to the compound of Formula (VII) with desired R₄ and R₅ in a multiple steps process using the commonly-known approaches in the literatures (Smith M., et al, Compendium of Organic Synthetic Methods, Smith M., et al ed. in 12 volumes, John Wiley & Sons, New York; Advanced Organic Chemistry, 5 ed. M. Smith & J. March, John Wiley & Sons, New York, 2001). Subsequently, the amino group of compound of Formula (VII) is converted to the appropriate pyrimidine-annulated triazole of Formula (I) in a multi-step process using the methods as described in Scheme 1.

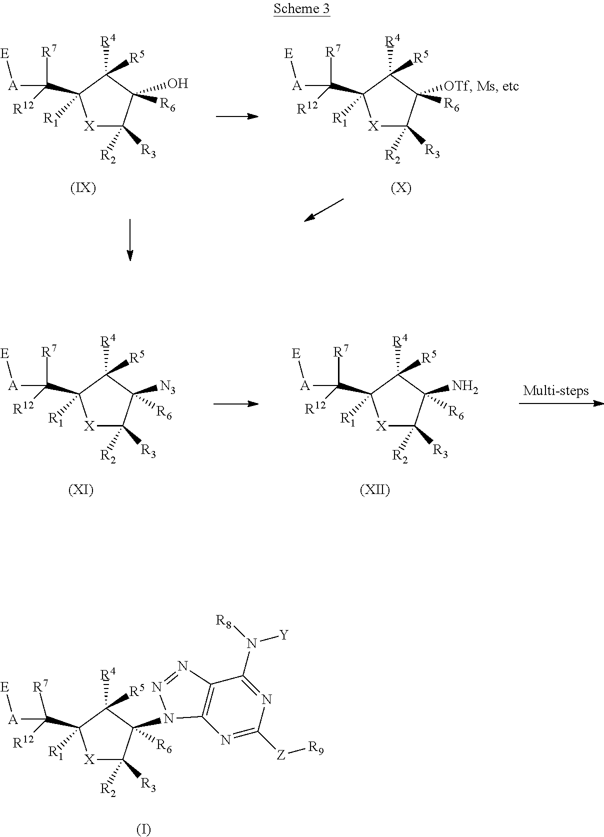

Scheme 3 illustrated yet another synthetic strategy for the preparation of compounds according to Formula (I). The appropriate five-membered heterocycles with a hydroxyl group on 3-position, Formula (IX), wherein R₁, R₂, R₃, R₄, R₅, R₆, R₇, R₁₂, X, A and E are as defined for Formula (I) and are optionally properly-protected wherever necessary, was converted to a sulfonylate such as mesylate, tosylate or triflate and so on and the resulting compounds of Formula (X) is subjected to a nucleophilic substitution by an azide to give compounds of Formula (XI) (Kenneth A. et al, J.Med.Chem. (1995), 38, 1720; Taichi K. eatl., Chemical Communications, 2012, 48(56), 7038; B. Lv et al., Tetrahedron Letters, 2018 59, 1473, Chen Y.D. et al, CN111333598). Alternatively, compounds of Formula (IX) are subjected to a Mitsunobu reaction conditions in the presence of an azide to directly yield compounds of Formula (XI) (Mitsunobu, O., Synthesis, 1981, 1; Green, J. et al, Org. Lett. 2009, 11, 807; G. Gosselin et al., Nucleosides and nucleotides, 1998, 17, 1731). The azido group of the resulting compound of Formula (XI) can be reduced to amine followed by the further conversion to the appropriate pyrimidine-annulated triazole of Formula (I) as described Scheme 1.

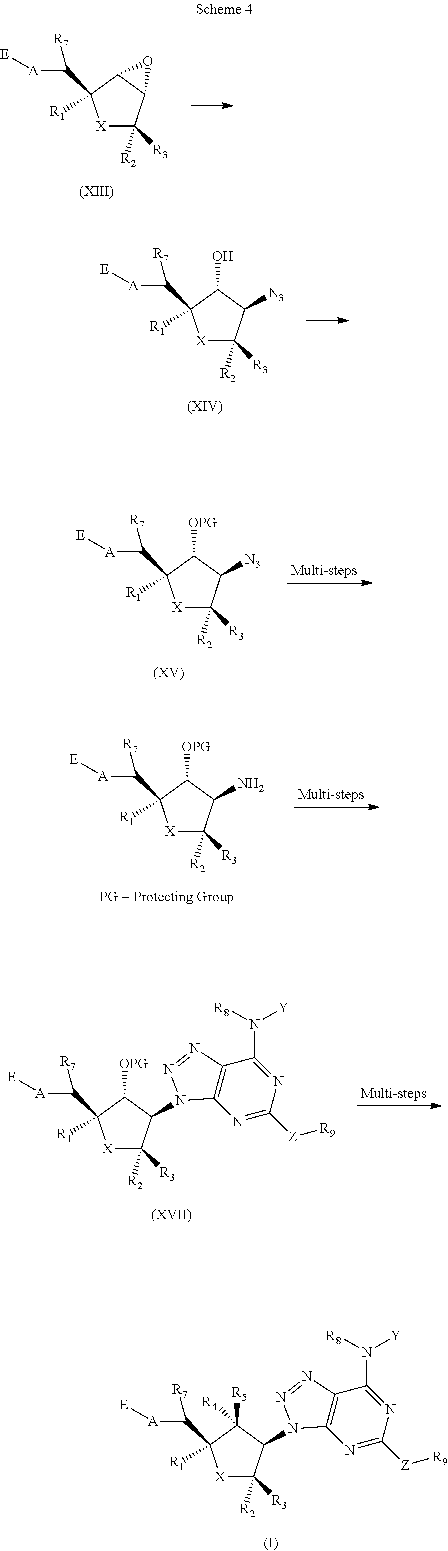

Scheme 4 described an alternative general method of preparation of compounds of general Formula (1) wherein R₆ is a hydrogen and R₁₂ is a hydrogen; The appropriate five-membered heterocylic epoxide, Formula (XIII), wherein R₁, R₂,R₃, R₇, X, A and E are as defined for Formula (1) and are optionally property-protected if necessary, are reacted with an azide, such as potassium azide, sodium azide, tretrabutylammoniumazide and so on. Afer the hydroxyl was protected with an appropriate protecting group, the azido group of the resulting compound of Formula (XV) can be reduced to amine followed by conversion to the appropriate pyrimidine-annulated triazole of Formula (XVIII) in several steps described in previous schemes. Finally desired R₄ and R₅ were introduced in a multiple steps process using the commonly-known approaches (Smith M., et al, Compendium of Organic Synthetic Methods, Smith M., et al ed. in 12 volumes, John Wiley & Sons, New York; Advanced Organic Chemistry, 5 ed. M. Smith & J. March, John Wiley & Sons, New York, 2001; Koushi H.et al, J. Med. Chem. 2018, 61, 5140; B. Lv et al., Tetrahedron Letters 2018, 59, 1473).

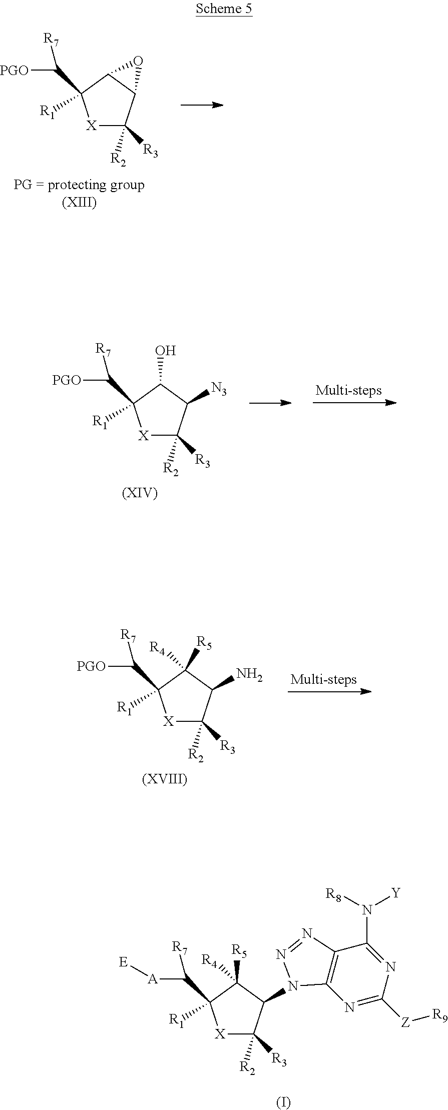

Scheme 5 describes an alternative general method for the preparation of compounds according to Formula (I) wherein R₆ is an hydrogen and R₁₂ is a hydrogen. The appropriate five-membered heterocylic epoxide, Formula (XVI), wherein R₁, R₂, R₃, R₆, R₇, X, A and E are defined for Formula (I) and are optionally properly-protected if necessary, are reacted with an azide, such as potassium azide, sodium azide, tretrabutylammoniumazide and so on, in an suitable solvent and a mixture of solvents. The resulting heterocylic azide can be converted to the compound of Formula (XVIII) with desired R₄ and R₅ in a multiple steps process using the common-known approaches in the literatures (Smith M., et al, Compendium of Organic Synthetic Methods, Smith M., et al ed. in 12 volumes, John Wiley & Sons, New York; Advanced Organic Chemistry, 5 ed. M. Smith & J. March, John Wiley & Sons, New York, 2001; Koushi H. et tl, J. Med. Chem. 2018, 61, 5140). Subsequently, the amino group of compound of Formula (XVIII) is converted to the appropriate pyrimidine-annulated triazole of Formula (I) in a multi-step process using the methods well known to the organic chemists versed in the art.

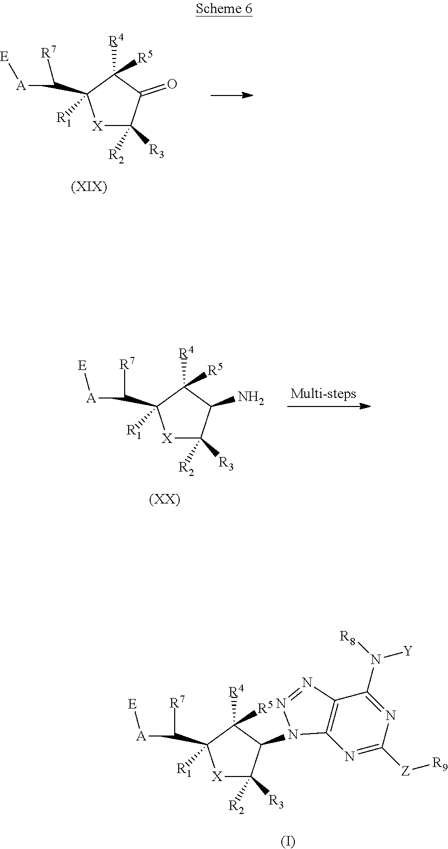

Scheme 6 illustrated another method for the preparation of compounds according to Formula (I) wherein R₆ is an hydrogen and R₁₂ is a hydrogen. The appropriate 3-oxo five-membered heterocycles of Formula (XIX), wherein R₆ is an hydrogen, R₁₂ is a hydrogen and R₁, R₂, R₃, R₄, R₅, R₇, X, A and E are as defined for Formula (I) and are optionally properly-protected if necessary, was subjected a reductive amination (B. Unterhait etal., Seientia Pharmaceutiea, 2000, 68, 102; Michael Aaron B. etal, US2018/208608; Michael J. etal, U.S. Pat. No. 6,355,660; Dong Bo Li etal, Org. Lett., 2013, 15(18), 4767) or transaminase catalyzed reaction (Matteo P. et al, Chem. Sci., 2019, 10, 5955) to afford the amine compound (XX), followed by the further conversion to the appropriate pyrimidine-annulated triazole of Formula (I) as described in above Schemes.

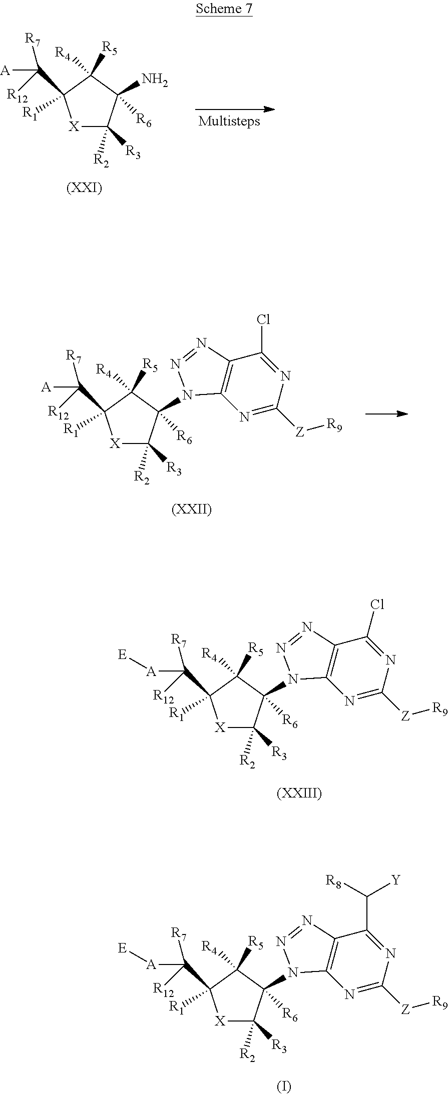

Scheme 7 describes an yet another alternative approach to prepare the compounds of Formula (I). Compounds of Formula (XXI), wherein R₁, R₂, R₃, R₄, R₅, R₆, R₁₂, R₇, X are as defined for Formula (I) and A is as defines as for Formula (I) or A is a functional group which can be transformed to A as defined for Formula (I). As mentioned in above schemes, Compounds of Formula (XXI) are optionally properly-protected if necessary. Compounds of Formula (XXI) are converted to Compounds of Formula (XXII) in a multi-steps reaction using the methods illustrated in above Shemes. The compounds of Formula (XXIII) are subsequently transformed in Compounds of Formula (XXIII) with desired E and A as define according to Formula (I) using the known methodology for functional group transformation and derivatization in literature of organic synthetic chemistry (Smith M., et al, Compendium of Organic Synthetic Methods, Smith M., et al ed. in 12 volumes, John Wiley & Sons, New York). Compounds of Formula (XXIII) are then further modified to provide Compounds of Formula (I) using the methods described in above Schemes.

### Examples

The examples and preparation provided below further illustrate and exemplify the compounds of this disclosure and methods of preparing such compounds. It is to be understood that the scope of this disclosure is not be limited in this way by the scope of the following examples and preparation.

The naming of the compounds in this document was made using the program “ChemDraw19.0” provided by Cambridge Scientific Computing Inc. If there is any inconsistency between the chemical name of the exemplified chemical compound and corresponding structure of said example, then the chemical structure should be used for determining the chemical compound of said example.

### Abbreviations

DIPEA N,N-diisopropylethylamine;
DMAP 4-dimethylaminopyridine;
DBU 2,3,4,6,7,8,9,10-octahydropyrimidol[1,2-a]azepine;
EtOAc ethyl acetate;
Et₃N triethylamine;
THF tetrahydrofuran;
DMF N,N-dimethylformamide;
DCM dichloromethane;
MS (ESI) mass spectroscopy (Electron Spray Ionization);
MeCN acetonitrile;
RT room temperature;
TBDPS t-butyldiphenylsilyl;
TBDPSCl t-butyldiphenylsilyl chloride;
TBAF tetrabutylammonium fluoride;
TFA trifluoroacetic acid;
DAST (diethylamino) sulfur trifluoride;
Ms Methanesulfonyl;
Ts p-Toluenesulfonyl;
Tf Trifluoromethanesulfonyl;
MeOH methanol;
BIOH ethanol;
AcOH acetic acid;
Me methyl;
Ac₂O acetic anhydride;
Tf₂O triflic anhydride;
AlBN azobisisobutyronitrile
aq aqueous
sat saturated
FBS Fetal bovine serum
PBS phosphate buffered saline

### Example 1

### The Preparation of ((2R,3R,4R,5S)-3-(7-(((1R,2S)-2-(3,4-difluorophenyl)cyclopropyl)amino)-5-(propylthio)-3H-[1,2,3]triazolo[4,5-d]pyrimidin-3-yl)-4-hydroxytetrahydrofuran-2,5-diyl)dimethanol (Compound 8)

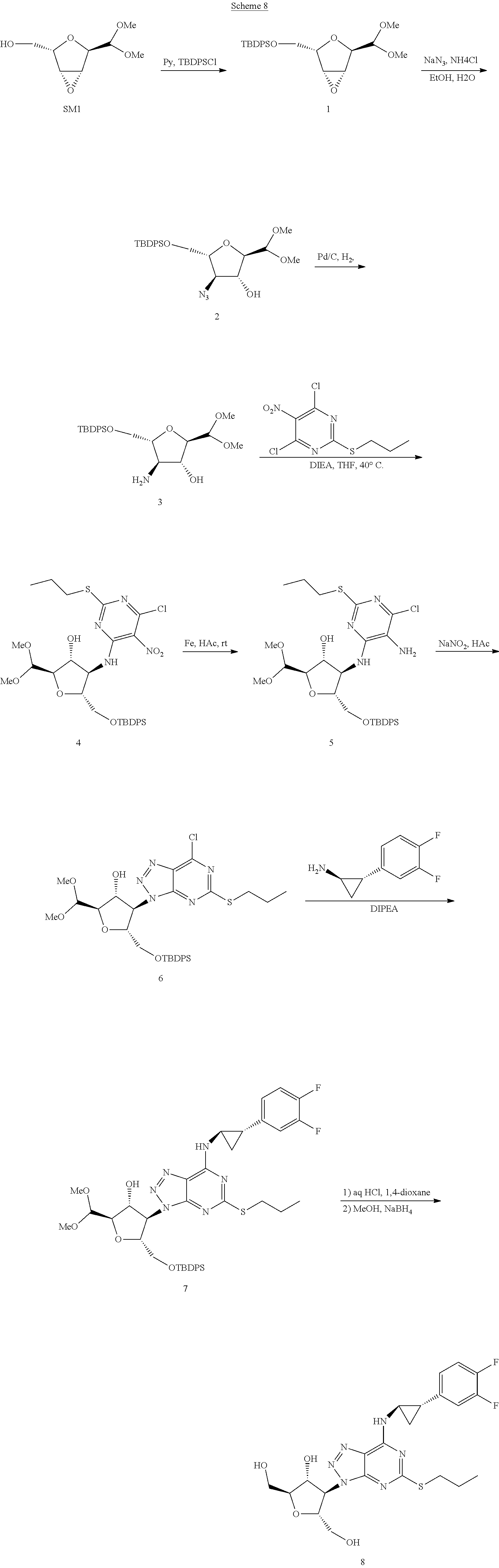

### Tert-butyl(((1R,2S,4R,5R)-4-(dimethoxymethyl)-3,6-dioxabicyclo[3.1.0]hexan-2-ylmethoxy)diphenylsilane (Compound 1)

A mixture of compound SM1 (910.00 mg,4.79 mmol), DMAP (1.17 g,9.58 mmol) and TBDPSCl (1.97 g,7.20 mmol) in dichloromethane (30 ml) was stirred at room temperature for 23 h. After that, it was washed with sat. aq NaHCO₃ (50 ml) and brine (50 ml), dried over anhydrous Na₂SO₄ and evaporated under reduced pressure. The residue was purified by column chromatography (hexane/ethyl acetate,8:1) to give compound 1 (2.00 g). MS (ESI) m/z: 446 [M+NH₄]⁺.

### (2R,3R,4R,5R)-4-azido-5-(((tert-butyldiphenylsilyl)oxy)methyl)-2-(dimethoxymethyl)tetrahydrofuran-3-ol (Compound 2)

A mixture of compound 1 (2.00 g, 4.67 mmol), sodium azide (2.72 g, 41.84 mmol) and ammonium chloride (2.75 g, 51.41 mmol) in water (12 ml) and ethanol (60 ml) was heated under reflux for two days. The mixture was concentrated to ⅓ volume, diluted with water(50 ml) and extracted with dichloromethane (100 ml×2). The organic layer was dried over Na₂SO₄, filterd and concentrated. The residue was purified by column chromatography (ethyl acetate/petroleum, 1:12) to give compound 2(1.50 g) as an oil. MS (ESI) m/z: 489 [M+NH4+]⁺.

### (2R,3R,4R,5R)-4-amino-5-(((tert-butyldiphenylsilyl)oxy)methyl)-2-(dimethoxymethyl)tetrahydrofuran-3-ol (Compound 3)

To a solution of compound 2 (50 mg, 0.11 mmol) in ethyl acetate (5 ml) was added Pd/C (20 mg) and the mixture was Hydrogenated under hydrogen overnight. The mixture was filtered and the filtrate was concentrated to give compound 3(46.00 mg). MS (ESI) m/z: 446[M+H]⁺

### (2R,3R,4R,5R)-5-(((tert-butyldiphenylsilyl)oxy)methyl)-4-((6-chloro-5-nitro-2-(propylthio)pyrimidin-4-yl)amino)-2-(dimethoxymethyl)tetrahydrofuran-3-ol (Compound 4)

A solution of compound 3 (370.00 mg, 0.83 mmol), 4,6-dichloro-5-nitro-2-(propylthio)pyrimidine(245.00 mg, 0.92 mmol) and triethylamine (251 mg,0.92 mmol) in tetrahydrofuran (10 ml) was stirred for 23 h. The mixture was diluted with ethyl acetate (50 ml) washed with brine (50 ml). The organic layer was dried over anhydrous Na₂SO₄ and evaporated, and the residue was purified by column chromatography (hexane/ethyl acetate, 5:1) to give compound 4 (440.00 mg). MS (ESI) m/z: 677 [M+H]⁺.

### (2R,3R,4R,5R)-5-(((tert-butyldiphenylsilyl)oxy)methyl)-4-(7-chloro-5-(propylthio)-3H-[1,2,3]triazolo[4,5-d]pyrimidin-3-yl)-2- (dimethoxymethyl)tetrahydrofuran-3-ol (Compound 6)

To a solution of compound 4 (440.00 mg,0.65 mmol) in acetic acid (9 ml) and water (2 ml) was added Fe powder (182.00 mg,3.26 mmol) and the mixture was stirred for 16 h at room temperature. (compound 5 was produced and used without purification). Then sodium nitrite (90.00 mg, 1.30 mmol) was added and the mixture was stirred at room temperature for 2 h. After that, the mixture was partitioned between ethyl acetate (200 ml) and sat.aq NaHCO₃ (200 ml). The organic layer was washed with brine (200 ml), dried over anhydrous Na₂SO₄, filtered and evaporated. The residue was purified by column chromatography (hexane/ethyl acetate,4:1) to give compound 6(300.00 mg). MS (ESI) m/z: 658 [M+H]⁺.

### (2R,3R,4R,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)-2-(dimethoxymethyl)tetrahydrofuran-3-ol (Compound 7)

A solution of (1R,2S)-2-(3,4-difluorophenyl)cyclopropanamine (147 mg, 0.87 mmol), compound 6(300 mg, 0.46 mmol) and DIPEA (119.00 mg, 0.92 mmol) in ethyl acetate (20 ml) was stirred at room temperature for 20 min. The solution was washed with 1N aq HCl (20 ml) sat.aq. NaHCO₃ (20 ml) and brine (20 ml), dried over Na₂SO₄ and evaporated. The residue was purified by column chromatography (hexane-ethyl acetate, 3:1) to give compound 7 (302.00 mg). ES-API m/z: 791 [M+H]⁺.

### ((2R,3R,4R,5S)-3-(7-(((1R,2S)-2-(3,4-difluorophenyl)cyclopropyl)amino)-5-(propylthio)-3H-[1,2,3]triazolo[4,5-d]pyrimidin-3-yl)- 4-hydroxytetrahydrofuran-2,5-diyl)dimethanol (Compound 8)

Compound 7(150.00 mg, 0.19 mmol) was dissolved in 1,4-dioxane (5ml) and 2% aq.HCl (5ml) was added. The reaction solution was heated at 90° C. for 3 h. After being cooled to room temperature, and the mixture was neutralized with sat.aq NaHCO₃ (PH8) and extracted with ethyl acetate (50 ml). The combined organic phase was washed with saturated brine (50 ml), dried over anhydrous Na₂SO₄ and evaporated under reduced pressure. The residue (150 mg) was dissolved in methanol (10 ml), sodium borohydride (100 mg, 2.64 mmol) was added and the solution was stirred at room temperature for 2 h. The solution was partitioned between ethyl acetate (50 ml) and water (50 ml). The organic layer was dried over anhydrous Na₂SO₄, filtered and evaporated. The residue was purified by column chromatography (dichloro methane/methanol,15:1) to give compound 8 (58.00 mg). MS (ESI) m/z: 509 [M+H]⁺. ¹H-NMR (400 MHZ, DMSO-d6): 9.137;(1H,d), 7.228-7.450;(3H, m), 5.132-5.620;(1H,m),4.624-5.110;(3H,m),3.746-4.175;(3H,m,),3.386-3.584;(4H,m),2.801-3.144;(3H,m),2.050-2.119;(1H,m), 1.377-1.821;(4H,m),0.815-0.996;(3H,m).

### Example 2

### The Preparation of ((2R,3R,5R)-3-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2,5-diyl)dimethanol (Compound 19)

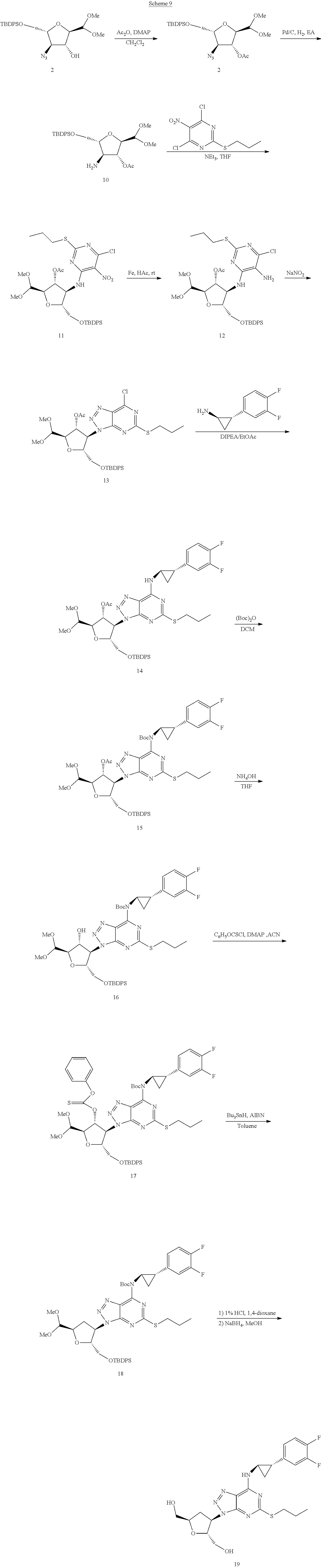

### (2R,3R,4S,5R)-4-azido-5-(((tert-butyldiphenylsilyl)oxy)methyl)-2-(dimethoxymethyl)tetrahydrofuran-3-yl acetate (Compound 9)

A solution of compound 2 (600.00 mg,1.27 mmol), DMAP (728.00 mg,5.96 mmol) and Ac₂O (510.00 mg,5.00 mmol) in dichloromethane (15 ml) was stirred at room temperature for 2 h. The solution was partitioned between dichloromethane and water The organic layer was washed with brine (20 ml), dried over Na₂SO₄ and evaporated. The residue was purified by column chromatography (hexane/ethyl acetate,8:1) to give compound 9 (650.00 mg). MS (ESI) m/z: 531 [M+NH₄]⁺.

### (2R,3R,4S,5R)-4-amino-5-(((tert-butyldiphenylsilyl)oxy)methyl)-2-(dimethoxymethyl)tetrahydrofuran-3-yl acetate (Compound 10)

A solution of compound 9 (650.00 mg, 1.27 mmol) in ethyl acetate (10 ml) was stirred with Pd/C (20%, 130 mg) under hydrogen overnight. After that, the mixture was filtered through celite and evaporation to give compound 10 (610.00 mg). MS (ESI) m/z: 488 [M+H]⁺.

### (2R,3R,4S,5R)-5-(((tert-butyldiphenylsilyl)oxy)methyl)-4-((6-chloro-5-nitro-2-(propylthio)pyrimidin-4-yl) amino)-2-(dimethoxymethyl)tetrahydrofuran-3-yl acetate (Compound 11)

A solution of compound 10 (610.00 mg,1.25 mmol) NEt3 (190 mg,0.10 mmol) and 4,6-dichloro-5-nitro-2-(propylthio) pyrimidine (403.00 mg,1.51 mmol) in THF(10 ml), was stirred at room temperature for 3 h. The mixture was partitioned between dichloromethane (50 ml) and sat. aq NaHCO₃ (50 ml). The organic layer was washed with brine (50 ml), dried over Na₂SO₄ and evaporated. The residue was purified by column chromatography (hexane/ethyl acetate,10:1) to give compound 11(750.00 mg). MS (ESI) m/z: 719 [M+H]⁺.

### (2R,3R,4S,5R)-5-(((tert-butyldiphenylsilyl)oxy)methyl)-4-(7-chloro-5-(propylthio)-3H-[1,2,3]triazolo[4,5-d]pyrimidin-3-yl)-2- (dimethoxymethyl)tetrahydrofuran-3-yl acetate (Compound 13)

A solution of compound 11 (750.00 mg,0.10 mmol) in acetic acid (10 ml) and water (2.5 ml) was stirred with Fe powder (292.00 mg,5.23 mmol) at room temperature overnight. compound 12 was formed and used directly. Sodium nitrite (144.00 mg, 2.09 mmol) was added and the mixture was stirred at room temperature for 3 h. After that, it was neutralized with sat.aq NaHCO₃ (pH>7) and ethyl acetate (50 ml) were added. After filtration through celite, the filtrate was partitioned between ethyl acetate and water, and the aqueous phase was extracted with ethyl acetate (50 ml). The combined organic phase was washed with brine (100 ml), dried over anhydrous Na₂SO₄, filtered and evaporated to give compound 12 (610.00 mg). ES-API m/z: 700 [M+H]⁺.

### (2R,3R,4S,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)-2-(dimethoxymethyl)tetrahydrofuran-3-yl acetate (Compound 14)

A solutiong of (1R,2S)-2-(3,4-difluorophenyl)cyclopropanamine (198 mg, 1.17 mmol) and compound 13 (610.00 mg, 0.87 mmol) and DIPEA (376.00 mg, 2.91 mmol) in ethyl acetat (20 ml) and the solution was stirred at room temperature for 16 h. The solution was washed with sat.aq. NaHCO₃ (20 ml) and brine (20 ml), dried over Na₂SO₄, filtered and evaporated. The residue was purified by column chromatography (hexane/ethyl acetate, 4:1) to give compound 14 (660.00 mg). MS (ESI) m/z: 833 [M+H]⁺.

### (2R,3R,4S,5R)-4-(7-((tert-butoxycarbonyl)((1R,2S)-2-(3,4-difluorophenyl)cyclopropyl)amino)-5-(propylthio)-3H-[1,2,3]triazolo[4,5-d]pyrimidin-3-yl)-5-(((tert-butyldiphenyl silyl)oxy)methyl)-2-(dimethoxymethyl)tetrahydrofuran-3-yl acetate (Compound 15)

To a solution of compound 14 (660.00 mg, 0.79 mmol), DMAP (116.00 mg, 0.95 mmol) in dichloromethane (10 ml) was added (Boc)₂O (208.00 mg, 0.95 mmol) and the reaction solution was stirred at room temperature for 24 h. After that, it was washed with water (50 ml) and brine (50 ml), dried over Na₂SO₄ and evaporated. The residue was purified by column chromatography (hexane/ethyl acetate, 8:1) to give compound 15 (630.00 mg). MS (ESI) m/z: 933 [M+H]⁺.

### Tert-butyl (3-((2R,3R,4R,5R)-2-(((tert-butyldiphenylsilyl)oxy)methyl)-5-(dimethoxymethyl)-4-hydroxytetrahydrofuran-3-yl)-5-(propylthio)-3H-[1,2,3]triazolo[4,5-d]pyrimidin-7-yl)((1R,2S)-2-(3,4-difluorophenyl)cyclopropyl)carbamate (Compound 16)

Compound 15 (630.00 mg, 0.68mmol) was dissolved in THF (5 ml) and methanol (5 ml), ammonium (1 ml) was added and the solution was stirred at 50° C. for 8.5 h. After being cooled to room temperature, water (20 ml) was added and the mixture was extracted with ethyl acetate(40 ml×2). The organic layer was washed with brine (40 ml), dried over Na₂SO₄, fulterd and evaporated. The residue was purified by column chromatography (hexane/ethyl acetate,6:1) to give compound 16(530.00 mg). MS (ESI) m/z: 933 [M+H]⁺.

### Tert-butyl (1-((2R,3S,4R,5R)-2-(((tert-butyldiphenylsilyl)oxy)methyl)-5-(dimethoxymethyl)-4-((phenoxycarbonothioyl)oxy)tetrahydrofuran-3-yl)-6-(propylthio)-1H- [1,2,3]triazolo[4,5-c]pyridin-4-yl)((1R,2S)-2-(3,4-difluorophenyl)cyclopropyl)carbamate (Compound 17)

A mixture of compound 16 (530.00 mg,0.60 mmol), DMAP (727.00 mg,5.95 mmol) and phenyl thiochloroformate (513.00 mg,2.97 mmol) in MeCN (20ml) was heated at 70° C. for 5 h. After being cooled to room temperature and evaporation, the residue was partitioned between water (50 ml) and dichloromethane (100 ml) and the aqueous phase was exacted with dichloromethane(100 ml). The combined organic phase was washed with brine (100 ml), dried over Na₂SO₄ and evaporated. The residue was purified by column chromatography (hexane/ethyl acetate,8:1) to give compound 17 (360.00 mg). ES-API m/z: 1027 [M+H]⁺.

### Tert-butyl (3-((2R,3R,5R)-2-(((tert-butyldiphenylsilyl)oxy)methyl)-5-(dimethoxymethyl)tetrahydrofuran-3-yl)-5-(propylthio)-3H-[1,2,3]triazolo[4,5-d]pyrimidin-7-yl)((1R,2S)-2-(3,4-difluorophenyl)cyclopropyl)carbamate (Compound 18)

Compound 17 (360.00 mg, 0.3 5mmol) was dissolved in toluene (5 ml), AIBN (73.00 mg, 0.44 mmol) and Bu₃SnH (306.00 mg, 1.05 mmol) were added, and the mixture was heated at 90° C. for 4 h. After being cooled to room temperature and evaporation, the residue was partitioned between sat. aq NaHCO₃ (50 ml) and dichloromethane (100 ml). The organic phase was washed with brine (50 ml), dried over Na₂SO₄, filtered and evaporated. The residue was purified by column chromatography (hexane-ethyl acetate, 10:1) to give compound 18(230.00 mg). MS (ESI) m/z: 875 [M+H]⁺.

((2R,3R,5R)-3-(7-(((1R,2S)-2-(3,4-difluorophenyl)cyclopropyl)amino)-5-(propylthio)-3H-[1,2,3]triazolo[4,5-d]pyrimidin-3- yl)tetrahydrofuran-2,5-diyl)dimethanol (Compound 19)

A solution of compound 18 (220.00 mg,0.25 mmol) in 1,4-dioxane (10 ml) and 2% HCl (10 ml) was heated at 90° C. for 3 h. After being cooled to room temperature, the mixture was neutralized with sat.aq NaHCO₃ (50 ml) and extracted with ethyl acetate (50 ml×2). The organic layer was washed with brine (100 ml), dried and evaporated under reduced pressure. The residue (195.00 mg) was dissolved in methanol (10 ml), sodium borohydride (26.00 mg, 0.69 mmol) was added and the solution was stirred at room temperature for 25 min. Water(50 ml) was added and the mixture was extracted with ethyl acetate (50 ml×2). The organic phase was washed with brine (50 ml), dried over Na₂SO₄, filtered and evaporated. The residue was purified by column chromatography (dichloromethane/methanol,20:1) to give compound 19 (23.00 mg).MS (ESI) m/z: 493 [M+H]⁺.¹H-NMR (400 MHZ,MeOH-d4): 7.21-7.07;(3H,m),5.38-5.35;(1H,q),4.56-4.52;(1H,m),4.40-4.4.36;(1H,m),3.70-3.80;(3H,m),3.62-3.56;(1H,dd),3.13-3.09;(1H,m),3.06-3.03;(1H,m),2.96-2.94;(1H,m),2.68-2.64;(2H,m),2.13-2.12;(1H,m),1.65-1.60;(2H,m),1.48-1.46;(1H,m),1.40-1.36;(1H,m),0.94-0.91;(3H,t).

### Example 3

### The Preparation of (2S,3R,4S)-4-(7-(((1R,2S)-2-(3,4-difluorophenyl)cyclopropyl)amino)-5-(propylthio)-3H-[1,2,3]triazolo[4,5-d]pyrimidin-3-yl)-2- (hydroxymethyl)tetrahydrofuran-3-ol (Compound 27)

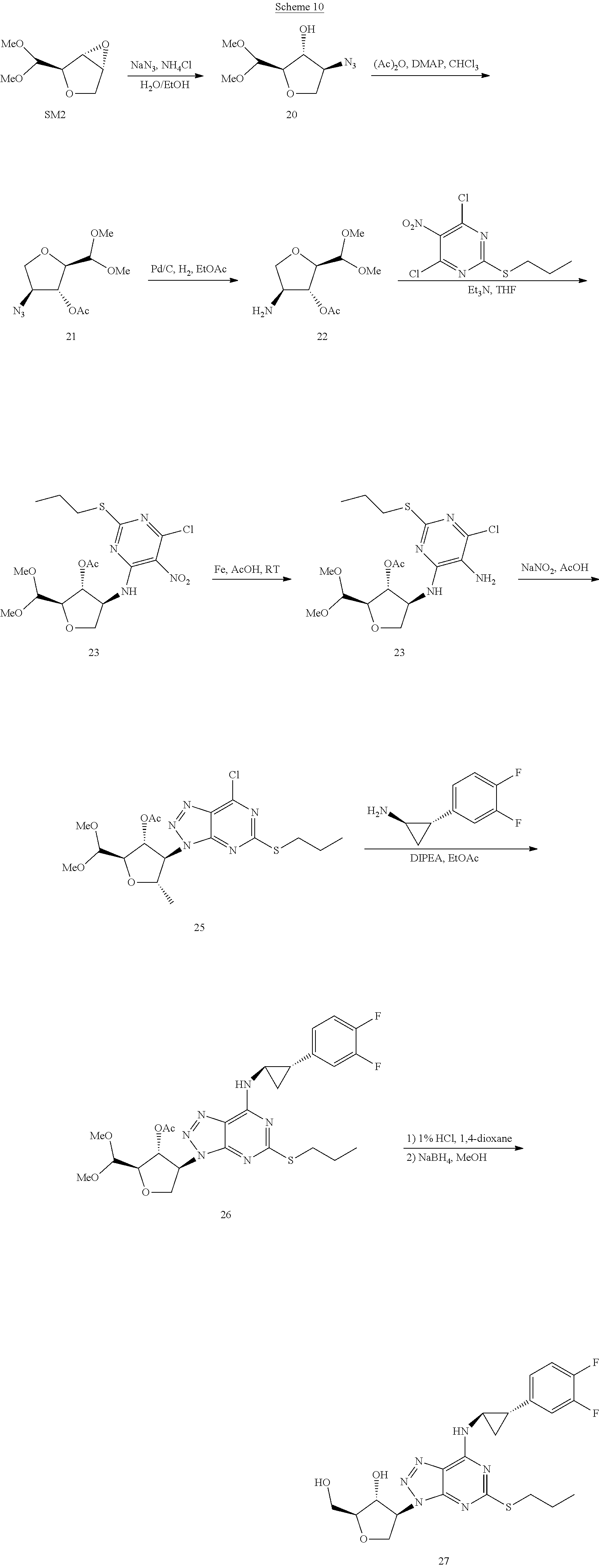

### (2R,3R,4S)-4-azido-2-(dimethoxymethyl)tetrahydrofuran-3-ol (Compound 20)

A mixture of compound SM2 (7.32 g, 45.56 mmol), sodium azide (11.89 g, 0.18 mol) and ammonium chloride (12.22 g, 0.23 mol) in water (73.2 ml) and ethanol (293 ml) was refluxed for 22 h. After cooling and evaporation, water (200 ml) was added and the mixture was extracted with dichloromethane (200 ml×2). The organic layer was washed with brine (200ml), dried overnight and evaporated to give compound 20 (7.77g). MS (ESI) m/z: 221 [M+NH₄]⁺.

### (2R,3R,4S)-4-azido-2-(dimethoxymethyl)tetrahydrofuran-3-yl acetate (Compound 21)

A solution of compound 20 (7.77 g,38.26 mmol), Ac₂O (15.60 g,0.15 mol) and DMAP (23.40 g, 0.19 mol) in 1,2-dichloroethane (200 ml) was heated at 55° C. for 3.5 h. After cooling, it was partitioned between dichloroethane and 1N aq. HCl(200 ml), the organic phase was further washed with sat.aq NaHCO₃(200 ml) and brine(200 ml), dried over anhydrous Na₂SO₄, filtered and evaporated. The residue was purified by column chromatography (hexane/ethyl acetate. 10:1) to give compound 21 (8.83 g). MS (ESI) m/z: 263 [M+NH₄]⁺.

### (2R,3R,4S)-4-amino-2-(dimethoxymethyl)tetrahydrofuran-3-ylacetate (Compound 22)

A solution of compound 21 (8.83 g, 36.03 mmol) in methanol (100 ml) was stirred with Pd/C (10%, 883.00 mg) under hydrogen overnight. After that, the mixture was filtered through celite and concentrated to give compound 22 (7.04 g). MS (ESI) m/z: 220 [M+H]⁺.

### (2R,3R,4S)-4-((6-chloro-5-nitro-2-(propylthio)pyrimidin-4-yl) amino)-2-(dimethoxymethyl)tetrahydrofuran-3-yl acetate (Compound 23)

4,6-dichloro-5-nitro-2-(propylthio) pyrimidine (6.75 g, 25.28 mmol) was dissolved in THF (135 ml), a solution of compound 22 (5.50 g, 25.10 mmol) in THF (110 ml) was added dropwise at 0° C., and the reaction mixture was stirred at room temperature for 17 h. After that, the mixture was evaporated and the residue was purified by column chromatography (hexane/ethyl acetate, 6:1) to give compound 23 (3.45 g). MS (ESI) m/z: m/z: 451 [M+H]⁺.

### (2R,3R,4S)-4-(7-chloro-5-(propylthio)-3H-[1,2,3]triazolo[4,5-d]pyrimidin-3-yl)-2-(dimethoxymethyl)tetrahydrofuran-3-yl acetate (Compound 25)

A solution of compound 23 (688.00 mg, 1.53 mmol) in acetic acid (5 ml) and water (1 ml) was stirred with Fe powder (427.00 mg, 7.65 mmol) at room temperature overnight (compound 24 was monitored by LC-MS), sodium nitrite (212.00 mg, 3.07 mmol) was added, and the mixture was further stirred for 1 h. ethyl acetate (50 ml) and water (50 ml) were added. The mixture was partitioned between ethyl acetate and water, and the aqueous phase was extracted with ethyl acetate (50 ml). The combined organic layer was washed with sat. aq. NaHCO₃ (50ml×2) and brine (50 ml), dried over Na₂SO₄, filtered and evaporated to give crude compound 25 (680.00 mg). MS (ESI) m/z: 432 [M+H]⁺.

### (2R,3R,4S)-4-(7-(((1R,2S)-2-(3,4-difluorophenyl)cyclopropyl)amino)-5-(propylthio)-3H-[1,2,3]triazolo[4,5-d]pyrimidin-3-yl)-2- (dimethoxymethyl)tetrahydrofuran-3-yl acetate (Compound 26)

A solution of (1R,2S)-2-(3,4-difluorophenyl) cyclopropanamine (264 mg, 1.56 mmol), compound 25(560.00 mg, 1.30 mmol) and DIPEA (504.00 mg, 3.90 mmol) in ethyl acetate (20 ml) was stirred at room temperature overnight. It was washed with sat.aq.NaHCO₃(50 m) and brine (50 ml), dried anhydrous Na₂SO₄, filtered and evaporated. The residue was purified by column chromatography (hexane/ethyl acetate,3:1) to give compound 26 (570.00 mg). MS (ESI) m/z: 565 [M+H]⁺.

### (2S,3R,4S)-4-(7-(((1R,2S)-2-(3,4-difluorophenyl)cyclopropyl)amino)-5-(propylthio)-3H-[1,2,3]triazolo[4,5-d]pyrimidin-3-yl)-2- (hydroxymethyl)tetrahydrofuran-3-ol (Compound 27)

A solution of Compound 28 (100.00 mg,0.18 mmol) in 1,4-dioxane (2 ml) and 2% aq HCl (2 ml) was heated at 85° C. for 2 h. After cooling, sat.aq NaHCO₃ (20 ml) was added, the mixture was extracted with ethyl acetate (40 ml×2) and the organic phase was washed with brine (40 ml), dried over anhydrous Na₂SO₄, filtered and evaporated under reduced pressure. The residue (108.00 mg,0.23 mmol) was dissolved in methanol (5 ml), sodium borohydride (15 mg) was added and the solution was stirred at room temperature for 20 min. Water (70 ml) was added and the mixture was extracted with ethyl acetate (50 ml× 2). The organic layer was washed with brine (50 ml), dried over anhydrous Na₂SO₄, filtered and evaporated. The residue was purified by column chromatography(DCM/Isopropanol, 10:1) to give compound 27 (40.00 mg). MS (ESI) m/z: 479 [M+H]⁺. ¹H-NMR (400 MHZ, MeOH-d₄): 7.09-7.26;(3H, m), 4.90-5.36;(1H, m), 4.81-4.84;(1H, t), 4.31-4.42;(2H, m), 3.74-3.96;(3H, m), 2.91-3.17;(3H, m), 2.13-2.21;(1H, m), 1.32-1.54;(4H, m),0.91-1.07;(3H,m).

### Example 4

### The Preparation of ((2R,4R)-4-(7-(((1S,2S)-2-(3,4-difluorophenyl)cyclopropyl)amino)-5-(propylthio)-3H-[1,2,3]triazolo[4,5-d]pyrimidin-3- yl)tetrahydrofuran-2-yl)methanol (Compound 32)

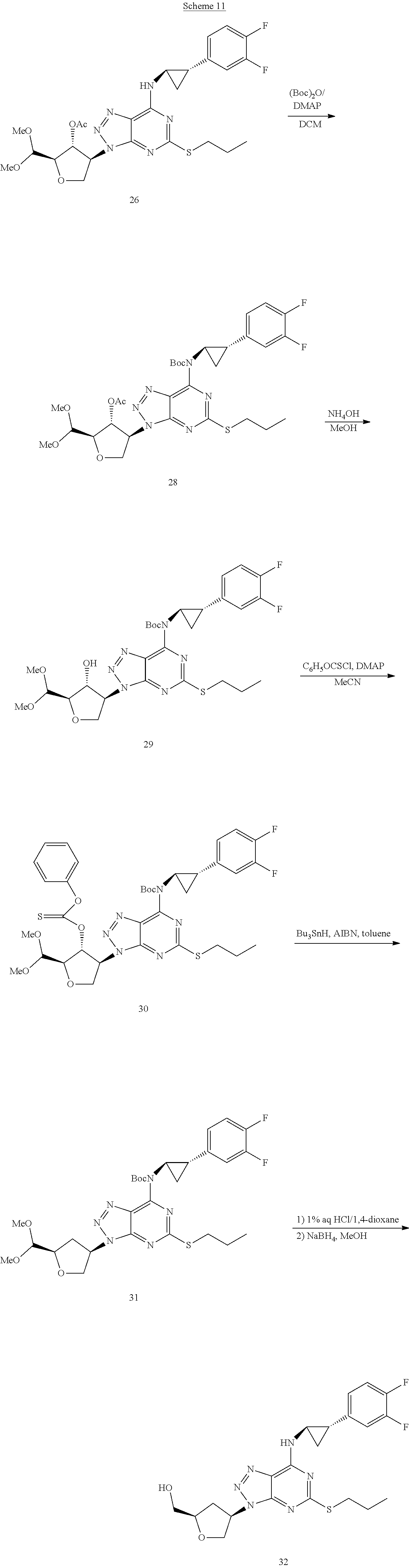

### (2R,3R,4S)-4-(7-((tert-butoxycarbonyl)((1R,2S)-2-(3,4-difluorophenyl)cyclopropyl)amino)-5-(propylthio)-3H-[1,2,3]triazolo[4,5-d]pyrimidin- 3-yl)-2-(dimethoxymethyl)tetrahydro furan-3-yl acetate (Compound 28)

To a solution of compound 26 (1.3g, 2.5 mmol) and DMAP (843 mg,6.9 mmol) in dichloromethane (20 ml) was added (Boc)₂O (754.00 mg, 3.46 mmol). The resulting solution was stirred at room temperature for 24 h. The solution was partitioned between dichloromethane and water (30 ml). The organic layer was washed with 1N aq HCl (10 ml), sat aq NaHCO₃(10 ml) and brine (30 ml), dried over anhydrous Na₂SO₄ and evaporated. The residue was purified by column chromatography on silica (dichloromethane-ethyl acetate, 10:1) to give compound 28 (1.317 g). MS (ESI) m/z: 665 [M+H]⁺.

### Tert-butyl ((1R,2S)-2-(3,4-difluorophenyl)cyclopropyl)(3-((3S,4R,5R)-5-(dimethoxymethyl)-4-hydroxytetrahydrofuran-3-yl)-5-(propylthio)-3H-[1,2,3]triazolo[4,5-d]pyrimidin-7-yl)carbamate (Compound 29)

To a solution of compound 28 (1.317 g, 1.98 mmol) was dissolved in THF (15 ml) and methanol (15 ml) was added NH₃·H₂O (41 ml). The reaction solution was stirred at. 50° C. for 8 h. After cooling and evaporation, water(20 ml) was added and the mixture was extracted with ethyl acetate (20 ml×2). The organic layer was washed with brine (25 ml), dried over Na₂SO₄ and evaporated under reduced pressure. The residue was chromatographed on silica, (dichloromethane-ethyl acetate,6:1) to give compound 29 (945 mg). MS (ESI) m/z: 623 [M+H]⁺.

Tert-butyl ((1R,2S)-2-(3,4-difluorophenyl)cyclopropyl)(1-((3S,4R,5R)-5-(dimethoxymethyl)-4-((phenoxycarbonothioyl)oxy)tetrahydrofuran-3-yl)-6- (propylthio)-1H-[1,2,3]triazolo[4,5-c]pyridin-4-yl)carbamate (Compound 30)

To a solution of compound 29(945.00 mg, 1.52 mmol) in MeCN(10 ml) was added DMAP (1.86 g,15.19 mmol) and phenyl thiochloroformate (1.31 g, 7.59 mmol) and the mixture was heated to 70° C. for 5 h. After cooling and evaporation, water (30 ml) was added and the mixture was extracted with DCM(25 ml×2). The organic layer was washed with brine (15 ml), dried over Na₂SO₄ and evaporated. The residue was chromatographed on silica, with N-hexane-ethyl acetate(5:1) as eluent, to give compound 30 (865 mg). MS (ESI) m/z: 758 [M+H]⁺.

### Tert-butyl((1R,2S)-2-(3,4-difluorophenyl)cyclopropyl)(3-((3R,5R)-5-(dimethoxymethyl)tetrahydrofuran-3-yl)-5-(propylthio)-3H-[1,2,3]triazolo[4,5-d]pyrimidin-7-yl)carbamate (Compound 31)

To a solution of compound 30(865 mg, 1.14 mmol) in toluene (20 ml) was added AIBN (187 mg, 1.14 mmol) and Bu₃SnH (830 mg, 2.85 mmol) and the mixture was stirred at 90° C. for 4 h. After cooling and evaporation, sat.aq. NaHCO₃ (50 ml) was added and the mixture was extracted with DCM (30 ml×2). The organic layer was washed with sat.aq NaHCO₃ (50 ml), dried over Na₂SO₄ and evaporated. The residue was chromatographed on silica (dichloromethane-ethyl acetate, 20:1) to afford compound 31 (646 mg). MS (ESI) m/z: 607 [M+H]⁺.

### ((2R,4R)-4-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2-yl)methanol (Compound 32)

To a solution of compound 31 (370 mg,0.61 mmol) in 1,4-dioxane (5 ml) was added 2% aq HCl (5 ml) and the mixture was heated to 90° C. for 2 h. After cooling, sat. aq.NaHCO3 (30ml) was added and the mixture was exacted with ethyl acetate(30 ml×2). The combined organic layer was washed with brine (30 ml), dried over Na₂SO₄ and evaporated under reduced pressure. The residue (280 mg) was dissolved in methanol (10 ml), sodium borohydride (16.00 mg,0.69 mmol) was added and the resulting mixture was stirred at room temperature for 30 min. The solution was partitioned between ethyl acetate (100ml) and water (50 ml). The organic layer was washed with brine (50 ml), dried over Na₂SO₄ and evaporated. The residue was chromatographed on silica (DCM/methanol, 40:1) to afford compound 32 (18.7 mg). MS (ESI) m/z: 463 [M+H]⁺. ¹H-NMR (400 MHZ, CDCl₃): 7.12-7.00;(3H, m), 6.77 (1H,s),5.52-5.48;(1H, m), 4.384.36;(1H,dd),4.35-4.27;(1H,m),4.20-4.17;(1H,m) 3.98-3.96;((1H,dd),3.79-3.76;(1H, dd), 3.12-3.06;(2H,m),2.99-2.95;(1H,m),2.68-2.62;(2H, m), 2.27-2.16;(1H, m), 1.68-1.64;(2H,m), 1.44-1.41;(1H,m), 1.38-1.35;(1H,m),0.96-0.93;(3H,t).

### Example 5

### The Preparation of ((2R,3S,4S,5S)-3-(7-(((1R,2S)-2-(3,4-difluorophenyl)cyclopropyl)amino)-5-(propylthio)-3H-[1,2,3]triazolo[4,5-d]pyrimidin-3-yl)-4-fluorotetrahydrofuran-2,5-diyl)dimethanol (Compound 39a)

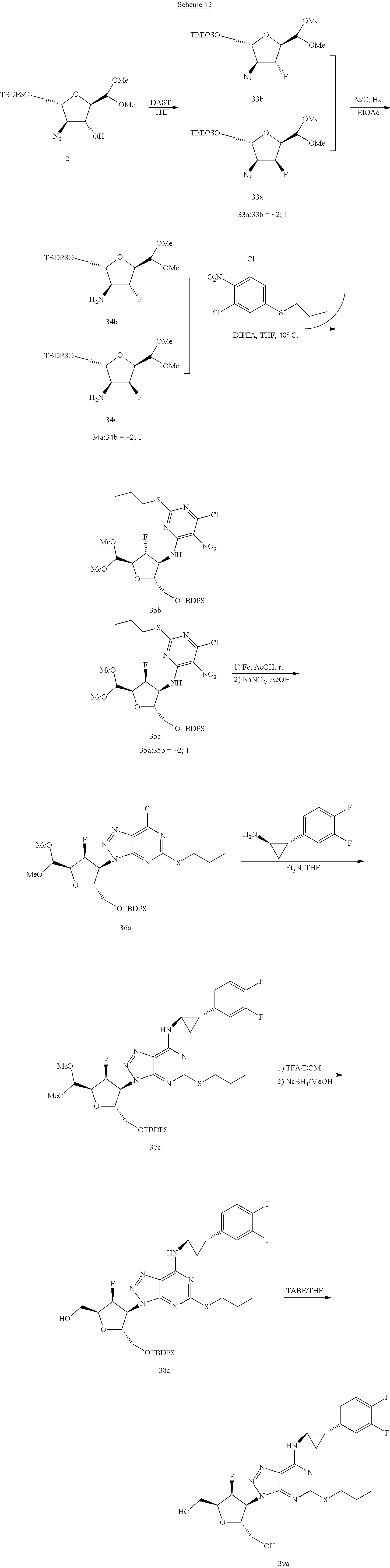

### (((2R,3S,4R,5S)-3-azido-5-(dimethoxymethyl)-4-fluorotetrahydrofuran-2-yl)methoxy)(tert-butyl)diphenylsilane (Compound 33a) and (((2R,3S,4S,5S)-3-azido-5-(dimethoxymethyl)-4-fluorotetrahydrofuran-2-yl)methoxy)(tert-butyl) diphenylsilane (Compound 33b)

To a solution of compound 2 (4.50 g, 9.55 mmol) in 50 ml THF was added DAST(3.08g, 19.1 mmol) and the reaction solution was stirred for 3 h under N₂. After being cooled to 0° C., it was neutralized by sodium saturated bicarbonate. The mixture was exacted with ethyl acetate (100 ml×2), the organic layer was washed with brine (100 ml), dried over anhydrous Na₂SO₄ and evaporated, the residue was purified by column chromatography on silica (N-hexane-ethyl acetate,10:1) to compound 33a and 33b (˜2:1, 2.38 g, 59.5%). MS (ESI) m/z: 491 [M+18]⁺.

### (2R,3S,4S,5S)-2-(((tert-butyldiphenylsilyl)oxy)methyl)-5-(dimethoxymethyl)-4-fluorotetrahydrofuran-3-amine (Compound 34a) and (2R,3S,4R,5S)-2-(((tert-butyldiphenylsilyl)oxy)methyl)-5-(dimethoxymethyl)-4-fluorotetrahydrofuran-3-amine (Compound 34b)

A solution of compound 33a and 33b (2.38 g, 5.03 mmol) in ethyl acetate (25 ml) was stirred with Pd/C (238 mg) under hydrogen overnight. Filtration through celite and evaporation gave compound 34a and 34b(2.00 g). MS (ESI) m/z: 448 [M+H]⁺.

### N-((2R,3S,4S,5S)-2-(((tert-butyldiphenylsilyl)oxy)methyl)-5-(dimethoxymethyl)-4-fluorotetrahydrofuran-3-yl)-6-chloro-5-nitro-2-(propylthio)pyrimidin-4- amine (Compound 35a) and N-((2R,3S,4R,5S)-2-(((tert-butyldiphenylsilyl)oxy)methyl)-5-(dimethoxymethyl)-4-fluorotetrahydrofuran-3-yl)-6-chloro-5-nitro-2-(propylthio)pyrimidin-4-amine (Compound 35b)

A solution of compound 34a and 34b (2.00 g, 4.47 mmol), 4,6-dichloro-5-nitro-2-(propylthio)pyrimidine (1.19 g,4.47 mmol) in tetrahydrofuran (45 ml) was stirred for 20 h. The solution was partitioned between ethyl acetate (50 ml) and water (40 ml). The organic layer was dried over anhydrous Na₂SO₄ and evaporated, and residue was chromatographed on silica(N-hexane-ethyl acetate, 25:1) to give compound 35a(1.24g) and compound 35b(690 mg: ). MS (ESI) m/z: 679 [M+H]⁺.

### 3-((2R,3S,4S,5S)-2-(((tert-butyldiphenylsilyl)oxy)methyl)-5-(dimethoxymethyl)-4-fluorotetrahydrofuran-3-yl)-7-chloro-5-(propylthio)-3H-[1,2,3]triazolo[4,5-d]pyrimidine (Compound 36a)

A solution of compound 35a (1.24 g, 1.83 mmol) in acetic acid (24 ml) and water (6 ml) was stirred with Iron powder (511.00 mg, 9.15 mmol) at room temperature. After 16 h, sodium nitrite(253.00 mg, 2.04 mmol) was added. After being stirred at room temperature for 2 h, the mixture was partitioned between ethyl acetate (200 ml) and sat aq. NaHCO₃ (200 ml). The organic layer was washed with brine (200 ml), dried over anhydrous Na₂SO₄ and evaporated in vacuum, and residue was chromatographed on silica(N-hexane-ethyl acetate, 10:1) to give compound 36a (540.00 mg). MS (ESI) m/z: 660 [M+H]⁺.

### 3-((2R,3S,4S,5S)-2-(((tert-butyldiphenylsilyl)oxy)methyl)-5-(dimethoxymethyl)-4-fluorotetrahydrofuran-3-yl)-N-((1R,2S)-2-(3,4- difluorophenyl)cyclopropyl)-5-(propylthio)-3H-[1,2,3]triazolo[4,5-d]pyrimidin-7-amine (Compound 37a)

A solution of (1R,2S)-2-(3,4-difluorophenyl)cyclopropanamine (78 mg, 0.455 mmol), compound 36a (250 mg, 0.379 mmol) and DIPEA (150.00 mg, 1.14 mmol) in ethyl acetate (20 ml) was stirred at room temperature for 1 h. It was partitioned between ethyl acetate (50 ml) and sat. aq. NaHCO₃ (20 ml). The organic layer was washed with sat.aq. NaHCO₃ (20 ml), dried over anhydros Na₂SO₄ and evaporated, and residue was chromatographed on silica (N-hexane-ethyl acetate, 5:1) as eluent, to give compound 37a (220.00 mg). ES (ESI) m/z: 793 [M+H]⁺.

### ((2S,3S,4S,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)-3-fluorotetrahydrofuran-2-yl) methanol (Compound 38a)

To a solution of compound 37a(220.00 mg, 0.278 mmol) in 23 ml dichloromethane was added trifluoroacetic acid (10 ml) and the reaction was stirred for 4 h at room temperature. Then it was poured into 300 ml sat. aq.NaHCO₃. After being stirred for 10 min, the mixture was extracted with dichloromethane (100 ml×2). The organic phase was washed with brine(100 ml), dried over anhydrous Na₂SO₄, filtered and concentrated under reduced pressure. The residue (207 mg) was dissolved in methanol (5 ml) and sodium borohydride (23 mg,0.55 mmol) was added. The solution was stirred at room temperature for 0.5 h. The solution was partitioned between ethyl acetate (50 ml) and water (50 ml). The organic layer was dried over anhydrous Na₂SO₄, filtered and evaporated under reduced pressure, and the residue was chromatographed on silica (n-hexane-ethyl acetate, 3:1) to give compound 38a (178.00 mg). MS (ESI) m/z: 749 [M+H]⁺.

### ((2R,3S,4S,5S)-3-(7-(((1R,2S)-2-(3,4-difluorophenyl)cyclopropyl)amino)-5-(propylthio)-3H-[1,2,3]triazolo[4,5-d]pyrimidin-3-yl)- 4-fluorotetrahydrofuran-2,5-diyl)dimethanol (Compound 39a)

To a solution of compound 43 (178 mg) in THF (5 ml) under inert atmosphere (N₂), was added TBAF (1M in THE, 1 ml) and the mixture was stirred at room temperature for 4 h. Then it was diluted with ethyl acetate (20 ml), and washed with water (10 ml×2) and brine (10 ml). The organic phase was dried over anhydrous Na₂SO₄, filtered and concentrated under reduced pressure. The residue was purified by silica column chromatography (dichloromethane/methanol, 40:11) to give compound 39a (36 mg). MS (ESI) m/z: 511 [M+H]⁺. ¹H-NMR (400 MHZ, CDCl₃): 7.14-6.98;(3H, m), 5.65;(0.5H, m), 5.55;(0.5H, m), 5.48-5.45;(0.5H, m), 5.39-5.37;(1.5H, m),4.60-4.57;(0.5H, m),4.52-4.50;(0.5H, m), 4.11-4.08;(1H,m),4.01-3.92;(2H,m),3.80-3.77;(1H,m),3.15-3.13;(1H,m),3.08-3.01;(2H,m), 2.96-2.91;(1H,m), 2.15-2.13;(1H,t), 1.66-1.61;(2H,m), 1.43-1.25;(2H,m),0.94-0.90;(3H,t).

### Example 6 The Preparation of ((2R,3S,4R,5S)-3-(7-(((1R,2S)-2-(3,4-difluoro phenyl)cyclopropyl)amino)-5-(propylthio)-3H-[1,2,3]triazolo[4,5-d]pyrimidin-3-yl)-4-fluorotetrahydrofuran-2,5-diyl)dimethanol (Compound 39b)

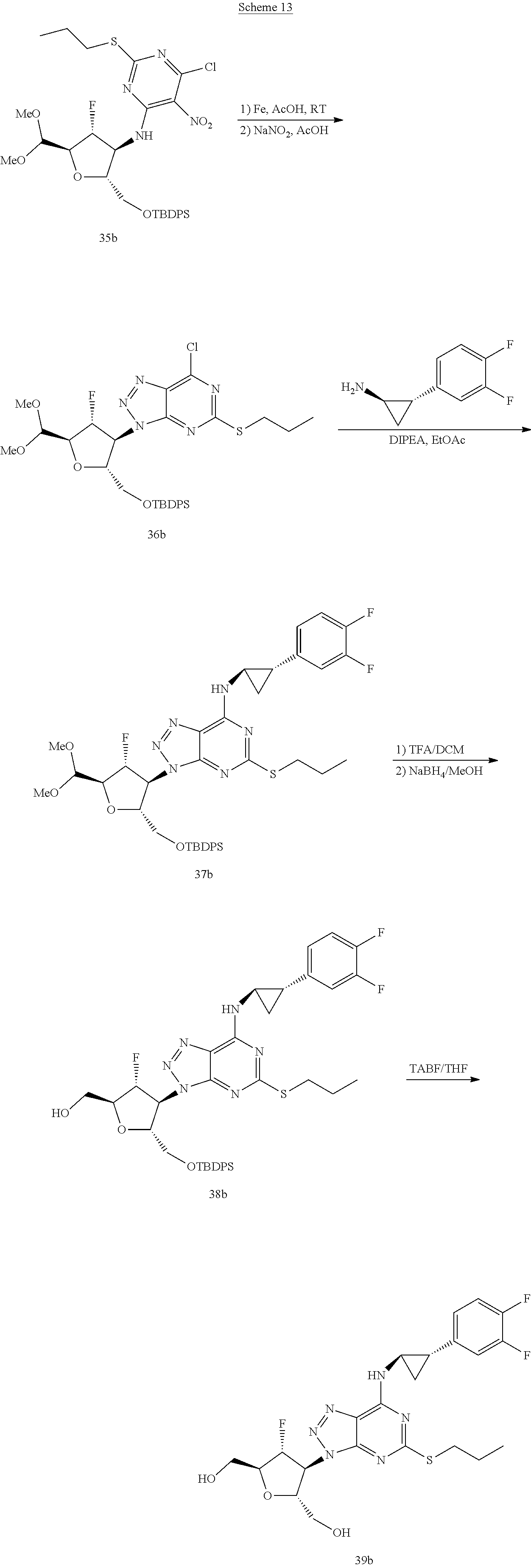

### 3-((2R,3S,4R,5S)-2-(((tert-butyldiphenylsilyl)oxy)methyl)-5-(dimethoxymethyl)-4-fluorotetrahydrofuran-3-yl)-7-chloro-5-(propylthio)-3H-[1,2,3]triazolo[4,5-d]pyrimidine (Compound 36b)

A solution of compound 35b(690.00 mg, 1.02 mmoll) in acetic acid (12 ml) and water (3 ml) was stirred with Iron powder (285.00 mg, 5.09 mmol) at room temperature. After 16 h, sodium nitrite (141.00 mg, 2.04 mmol) was added. The mixture was stirred at room temperature for additional 2 h. The mixture was partitioned between ethyl acetate (200 ml) and sat.aq.NaHCO₃ (200 ml). The organic layer was washed with brine (200 ml), dried over anhydrous Na₂SO₄ and evaporated under reduced pressure. The residue was chromatographed on silica gel(hexane-ethyl acetate, 10:1) to give compound 36b (280.00 mg, 41.72%). MS (ESI) m/z: 660 [M+H]⁺.

### 3-((2R,3S,4R,5S)-2-(((tert-butyldiphenylsilyl)oxy)methyl)-5-(dimethoxymethyl)-4-fluorotetrahydrofuran-3-yl)-N-((1R,2S)-2-(3,4- difluorophenyl)cyclopropyl)-5-(propylthio)-3H-[1,2,3]triazolo[4,5-d]pyrimidin-7-amine (Compound 37b)

A solution of (1R,2S)-2-(3,4-difluorophenyl)cyclopropanamine (62 mg, 0.364 mmol), compound 37b(200 mg, 0.303 mmol) and DIPEA (120.00 mg,0.91 mmol) in ethyl acetate (10 ml) was stirred at room temperature for 1 h. The solution was partitioned between ethyl acetate (50 ml) and sat. aq.NaHCO₃ (20 ml). The organic layer was washed with brine (20 ml), dried over anhydrous Na₂SO₄ and evaporated under reduced pressure. The residue was purified by silica gel column (n-hexane-ethyl acetate,5:1) give compound 37b(224.00 mg). MS (ESI) m/z: 793 [M+H]⁺.

### ((2S,3R,4S,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)-3-fluorotetrahydrofuran-2-yl)methanol (Compound 38b)

To a solution of (200.00 mg, 0.278 mmol) in 23 ml dichloromethane was added 10 ml of trifluoroacetic acid. The reaction was stirred for 4 h at room temperature. After that, the mixture was dropped to sat.aq. NaHCO₃(300 ml), stirred for additional 10 min and extracted with dichloromethane (2×100 ml). The organic phase was washed with brine (100 ml), dried with anhydrous Na₂SO₄, filtered and evaporated under reduced pressure. The residue (205 mg) was dissolved in methanol (5 ml), sodium borohydride (22 mg,0.55 mmol) was added, and the solution was stirred at room temperature for 0.5 h. The solution was partitioned between ethyl acetate (50 ml) and water (50 ml). The organic layer was dried and evaporated in vacuo. The residue was purified by silica gel column(n-hexane-ethyl acetate, 3:1) to give compound 38b (135.00 mg). MS (ESI) m/z: 749 [M+H]⁺.

### ((2R,3S,4R,5S)-3-(7-(((1R,2S)-2-(3,4-difluorophenyl)cyclopropyl)amino)-5-(propylthio)-3H-[1,2,3]triazolo[4,5-d]pyrimidin-3-yl)- 4-fluorotetrahydrofuran-2,5-diyl)dimethanol (Compound 39b)

To a solution of compound 38b (135 mg, 0.18 mmol) in 5 ml THF was added TBAF (1M in THF, 1 ml) and the mixture was stirred at room temperature for 4 h. Then it was partitioned between ethyl acetate (20 ml) and water(10 ml). The organic phase was washed with water (10 ml) and brine (10 ml), dried over anhydrous Na₂SO₄. filtered, and concentrated under reduced pressure. The residue was purified by slica gel column chromatography (dichloromethane/methanol, 40:1) to give compound 39b(26 mg). MS (ESI) m/z: 511 [M+H]^{+ 1}H-NMR (400 MHZ, CDCl₃): 7.63-6.98;(4H, m), 6.08-6.07;(0.5H,t),5.95-5.93;(0.5H, t), 5.64-5.58;(1H, m), 4.59-4.50;(1H, m),4.49-4.44;(1H, m),3.99-3.93;(3H, m), 3.73-3.69;(1H, m), 3.16-3.05; (2H,m),2.96-2.91;(1H,m),2.17-2.15;(1H, m), 1.66-1.61;(2H,m), 1.44-1.39;(2H,m), 1.05-1.01;(3H,t).

### Example 7

### The Preparation of (2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5-(hydroxymethyl)tetrahydrofuran-2-carboxylic acid (Compound 42)

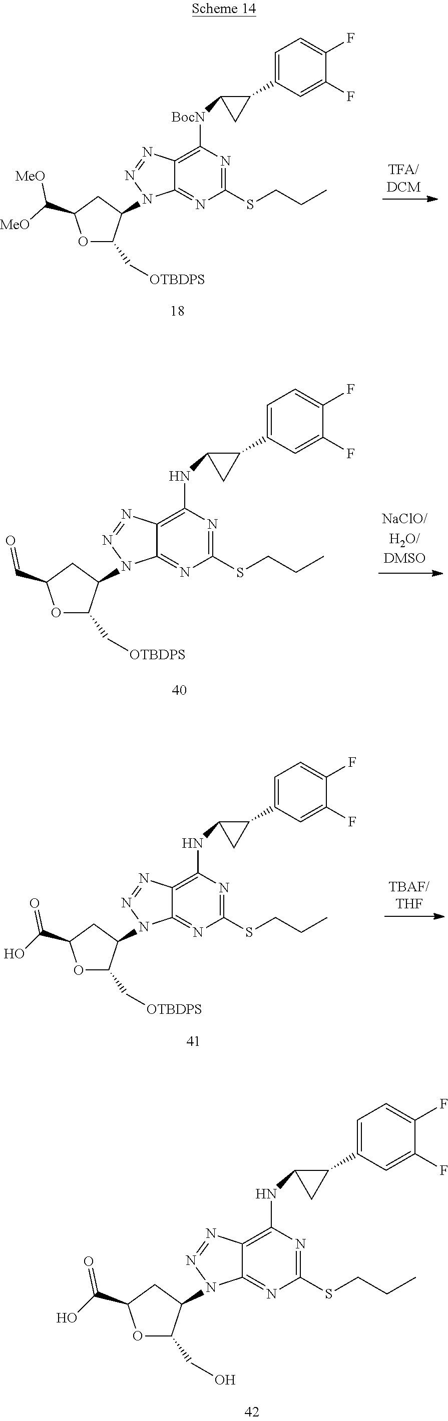

### (2R,4R,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2-carbaldehyde (Compound 40)

To a solution of compound 18 (960 mg, 1.10 mmol) in dichloromethane (15 ml) was added TFA (5 ml) and the reaction solution was stirred at room temperature for 3 h. After that, the mixture was dropped to sat.aq. NaHCO₃(100 ml) and extracted with dichloromethane (2×30 ml). The organic phase was washed with brine (100 ml), dried over anhydrous Na₂SO₄, filtered and evaporated under reduced pressure to afford compound 40(760 mg) as a light yellow foamy solid. MS (ESI) m/z: 747 [M+H₂O+H]⁺

### (2R,4R,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2-carboxylic acid (Compound 41)

To a solution of compound 40 (400 mg, 0.56 mmol) in a mixed solvent of THF (3 ml), DMSO (3 ml) and H₂O(3 ml) was added 0.2M aq.Na₂HPO₄ (2 ml) and NaClO(75 mg, 1.00 mmol) and the resulting mixture was stirred at room temperature for 1.5 h. The reaction was quenched with sat.aq Na₂S₂O₃(10 ml), adjusted to PH=4˜5 with IN aq.HCl and extracted with ethyl acetate(20 ml×3).The organic phase was washed with brine (20 ml), dried over anhydrous Na₂SO₄, filtered and evaporated under reduced pressure to afford compound 41 (400 mg) as a white foamy solid. MS (ESI) m/z: 745 [M+H]⁺.

### (2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5- (hydroxymethyl)tetrahydrofuran-2-carboxylic Acid (Compound 42)

To a solution of compound 41(400 mg, 0.54 mmol) in dry THF (10 ml) was added TBAF (1 ml, 1M in THF) and the resulting mixture was stirred at room temperature overnight. After that, the reaction mixture was diluted with ethyl acetate (50 ml) and washed with 1M aq. HCl (20 ml) and brine (20 ml). The organic phase was dried over anhydrous Na₂SO₄ and evaporated under reduced pressure. The residue was purified by C₁₈ column chromatography (acetonitrile/H₂O, 6:4˜5:5) to afford compound 42(110 mg) as a white foamy solid. MS (ESI) m/z: 507 [M+H]⁺. ¹H-NMR (400 MHZ, DMSO): 9.0;(1H,s),7.38-7.29;(2H,m), 7.09-7.07;(1H,m), 5.30-5.24;(4H,q),4.70-4.67;(1H, t), 4.55-4.46;(1H,m),3.61-3.57;(1H,dd), 3.50-3.3.46;(1H,dd),3.18-3.07;(2H,m),2.97-2.78;(3H,m),2.15-2.10;(1H,m),1.57-1.47;(2H,m),1.41-1.36;(1H,q),1.01-0.97;(1H,q),0.85-0.81;(3H,t).

### Example 8

### The Preparation of (2R,4R,5R)-4-(7-(((1R,2S)-2-(3,4-difluorophenyl)cyclopropyl)amino)-5-(propylsulfinyl)-3H-[1,2,3]triazolo[4,5-d]pyrimidin-3-yl)-5-(hydroxymethyl) tetrahydrofuran-2-carboxylic Acid (Compound 43)

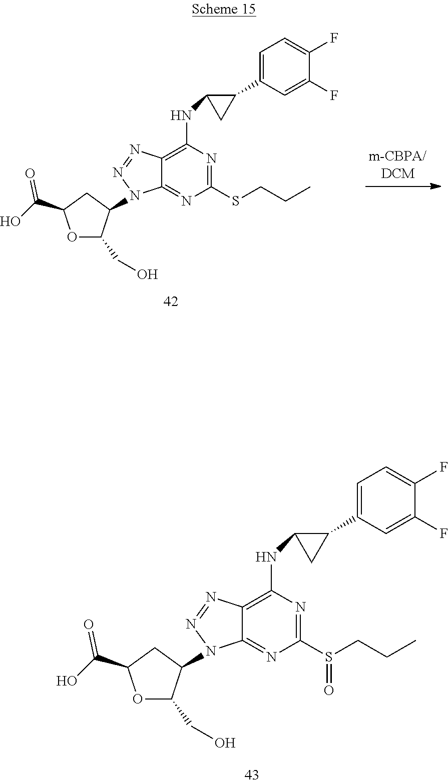 [SMILES: CCCSc1nc(N[C@@H]2C[C@H]2c2ccc(F)c(F)c2)c2nnn([C@@H]3C[C@H](C(=O)O)O[C@H]3CO)c2n1 ; CCCS(=O)c1nc(N[C@@H]2C[C@H]2c2ccc(F)c(F)c2)c2nnn([C@@H]3C[C@H](C(=O)O)O[C@H]3CO)c2n1]

To a solution of compound 42 (80 mg, 0.16 mmol) in DCM (20 ml) was added m-CPBA (27.3 mg, 0.16 mmol) and then the resulting mixture was stirred at room temperature for 5 h. The mixture was concentrated under reduced pressure and the residue was purified by C₁₈ column chromatography (acetonitrile/H₂O,1:5˜1:4) to afford compound 43(28 mg) as a white solid. MS(ESI) m/z: 523 [M+H]⁺. ¹H-NMR (400 MHZ, CDCl₃): 7.11-6.94;(3H,m), 5.62-5.58;(1H,m), 4.90-4.88;(1H,m),4.65-4.50;(1H, m), 3.97-3.93;(1H,dd), 3.77-3.74;(1H,dd), 3.30-3.26;(2H,m), 3.17-3.01;(3H,m),2.20-2.13;(1H,m),1.72-1.67;(2H,m),1.45-1.35;(2H,m),1.05-0.98;(3H,t).

### Example 9

### The Preparation of ((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5-(methoxymethyl)tetrahydrofuran-2-yl)methanol (Compound 49)

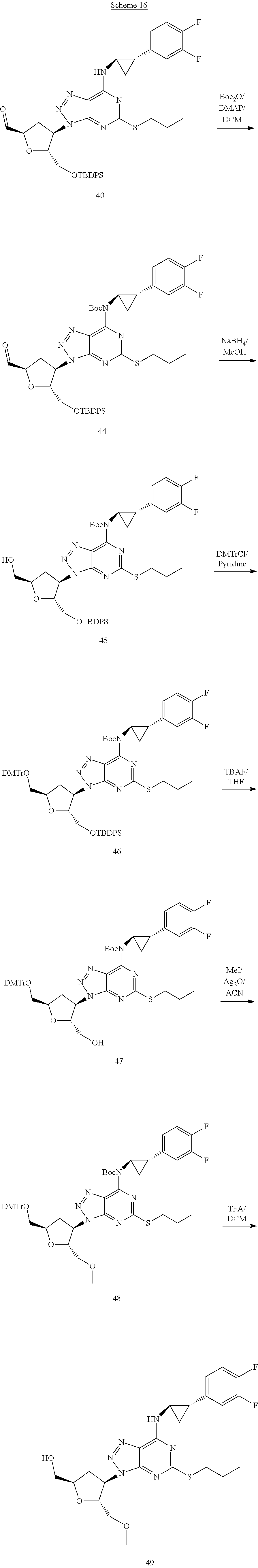

### Tert-butyl(3-((2R,3R,5R)-2-(((tert-butyldiphenylsilyl)oxy)methyl)-5-formyltetrahydrofuran-3-yl)-5-(propylthio)-3H-[1,2,3]triazolo[4,5-d]pyrimidin-7-yl)((1R,2S)-2-(3,4-difluorophenyl)cyclopropyl)carbamate (Compound 44)

To a solution of compound 40(850 mg, 1.14 mmol) in dichloromethane (50 ml) was added DMAP (310 mg, 2.54 mmol) and Boc₂O (300 mg, 1.37 mmol) and the resulting solution was stirred overnight. After that, the solution was washed with 1N aq. HCl (10 ml) and the organic phase was washed with brine (10 ml), dried over anhydrous Na₂SO₄, filtered and evaporated under reduced pressure to afford compound 44(960 mg). MS (ESI) m/z: 847 [M+H₂O+H]⁺.

### Tert-butyl (3-((2R,3R,5R)-2-(((tert-butyldiphenylsilyl)oxy)methyl)-5-(hydroxymethyl)tetrahydrofuran-3-yl)-5-(propylthio)-3H-[1,2,3]triazolo[4,5-d]pyrimidin-7-yl)((1R,2S)-2-(3,4-difluorophenyl)cyclopropyl)carbamate (Compound 45)

To a solution of compound 44(960 mg, 1.13 mmol) in methanol (20 ml) was added NaBH₄(55 mg, 1.44 mmol) and the resulting mixture was stirred at 0° C. for 1 h. After that, the mixture was partitioned between dichloromethane (60 ml) and water (60 ml). The organic phase was washed with brine (20 ml), dried over anhydrous Na₂SO₄ and filtered and evaporated under reduced pressure to afford compound 45(831 mg). MS (ESI) m/z: 831 [M+H]⁺.

### Tert-butyl (3-((2R,3R,5R)-5-((bis (4-methoxyphenyl)(phenyl)methoxy)methyl)-2-(((tert-butyldiphenylsilyl)oxy)methyl)tetrahydrofuran-3-yl)-5-(propylthio)-3H-[1,2,3]triazolo[4,5-d]pyrimidin-7-yl)((1R,2S)-2-(3,4-difluorophenyl)cyclopropyl)carbamate (Compound 46)

To a solution of compound 45 (831 mg, 1.00 mmol) in dried pyridine(20 ml) was added DMTrCl (507 mg, 1.50 mmol) and the resulting mixture was stirred at room temperature overnight. After that, the mixture was diluted with dichloromethane (50 ml) and washed with brine (30 ml×3). The organic phase was dried over anhydrous Na₂SO₄, filtered and evaporated under reduced pressure. The residue was further purified by column chromatography (n-hexane/ethyl acetate, 15:1˜5:1) to afford compound 46 (660 mg). MS (ESI) m/z: 1133 [M+H]⁺.

### Tert-butyl (3-((2R,3R,5R)-5-((bis (4-methoxyphenyl)(phenyl)methoxy)methyl)-2-(hydroxymethyl)tetrahydrofuran-3-yl)-5-(propylthio)-3H-[1,2,3]triazolo[4,5-d]pyrimidin-7-yl)((1R,2S)-2-(3,4-difluorophenyl)cyclopropyl)carbamate (Compound 47)

To a solution of compound 46(660 mg, 0.58 mmol) in THF (10 ml) was TBAF (2.5 ml, 1M in THF) and the reaction solution was stirred at room temperature for 3 h. After evaporation, the residue was purified by silica gel column chromatography (n-hexane/ethyl acetate, 3:1) to afford compound 47(190 mg). MS (ESI) m/z: 895 [M+H]⁺.

### Tert-butyl (3-((2R,3R,5R)-5-((bis (4-methoxyphenyl)(phenyl)methoxy)methyl)-2-(methoxymethyl)tetrahydrofuran-3-yl)-5-(propylthio)-3H-[1,2,3]triazolo[4,5-d]pyrimidin-7-yl)((1R,2S)-2-(3,4-difluorophenyl)cyclopropyl)carbamate (Compound 48)

A mixture of compound 47(145 mg, 0.16 mmol), MeI (350 mg,2.46 mmol) and Ag₂O (600 mg, 4.84 mmol) in acetonitrile (6 ml) was stirred overnight. After filtration, the filtered cake was washed with ethyl acetate (30 ml) and the filtrate was evaporated under reduced pressure to afford compound 48(100 mg). MS (ESI) m/z: 909 [M+H]⁺.

### ((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5- (methoxymethyl)tetrahydrofuran-2-yl)methanol (Compound 49)

To a solution of compound 48(100 mg, 0.11 mmol) in dichloromethane (10 ml) was TFA (2 ml) and the reaction solution was stirred for 1 h. After that, it was quenched with sat.aq NaHCO₃ (30 ml) and exacted with DCM (30 ml). The organic phase was dried over anhydrous Na₂SO₄, filtered and concentrated under reduced pressure. The residue was purified by silica gel column chromatography (n-hexane/ethyl acetate, 1:2) to afford compound 49(50 mg). MS (ESI) m/z: 507 [M+H]⁺. ¹H-NMR (400 MHZ, CDCl₃): 7.20-6.95;(3H,m), 6.68;(1H,s), 5.46-5.40;(1H,q), 4.67-4.64;(1H,m),4.50-4.47;(1H,m), 3.90-3.86;(1H,dd), 3.81-3.72;(2H,m), 3.63-3.60;(1H,dd), 3.52-3.48;(1H,m), 3.39;(3H,s), 3.07;(3H,m),2.81-2.75;(1H,m),2.67-2.60;(1H,m), 2.16-2.14;(1H,m), 1.68-1.66;(2H,m), 1.42-1.38;(2H,m), 0.99-0.96;(3H,t).

### Example 10

### The Preparation of 2-((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5-(hydroxymethyl)tetrahydrofuran-2-yl)ethan-1-ol (Compound 54)

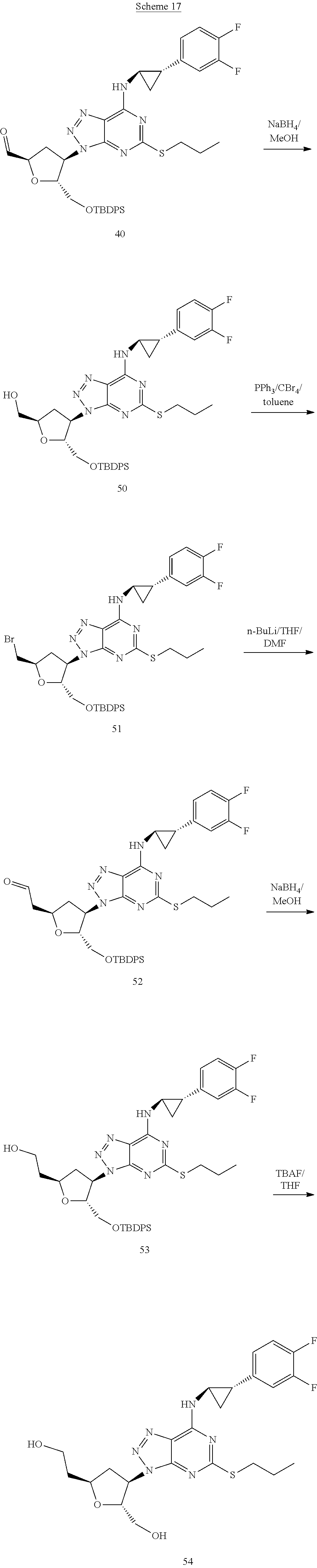

### ((2R,4R,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2-yl)methanol (Compound 50)

To a stirred solution of compound 40(1.50 g, 2.06 mmol) in methanol(30 ml) at 0° C. was added NaBH₄ (273 mg, 7.2 1mmol) and the reaction mixture was stirred for 4 h. After that, the reaction was quenched to PH=7 with 2% aq. HCl, concentrated and extracted with dichloromethane (100 ml×3). The organic layer was washed with brine (10 ml×2), dried over anhydrous Na₂SO₄, filtered and concentrated under reduced pressure. The residue was purified by silica gel column chromatography (n-hexane/ethyl acetate, 5:1 to 3:1) to afford compound 50 (900 mg). MS (ESI) m/z: 731 [M+H]⁺.

Tert-butyl (3-((2R,3R,5R)-5-(bromomethyl)-2-(((tert-butyldiphenylsilyl)oxy)methyl)tetrahydrofuran-3-yl)-5-(propylthio)-3H-[1,2,3]triazolo[4,5-d]pyrimidin-7-yl)((1R,2S)-2-(3,4-difluorophenyl)cyclopropyl)carbamate (Compound 51)

A solution of PPh₃(197 mg, 0.75 mmol) and CBr₄ (259 mg, 0.75 mmol) in toluene (20 ml) was stirred at 0° C. for 15 min. Compound 50 (500 mg, 0.68 mmol) was added and then the resulting solution was further stirred at room temperature for 2 h. After evaporation, the residue was purified by gel column chromatography (n-hexane/ethyl acetate, 5:1 to 3:1) to afford compound 51(300 mg). MS (ESI) m/z: 793 [M+H]⁺.

### 2-((2R,4R,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2-yl)acetaldehyde (Compound 52)

To a solution of compound 51(200 mg, 0.25 mmol) in THF (20 ml) under N₂ atmosphere at −78° C. was added n-BuLi(0.9 ml, 1.6M in THF) and the mixture was stirred at this temperature for 15 min. DMF (4.5 ml) was added and then the resulting mixture was stirred at room temperature for 1 h. The reaction was quenched with sat.aq.NH₄Cl (10 ml) and extracted with ethyl acetate (50 ml). The organic phase was collected and the aqueous phase was extracted with ethyl acetate (20 ml). The combined organic phase was washed with brine (10 ml×2), dried over anhydrous Na₂SO₄, filtered and concentrated under reduced pressure. The residue was further washed with n-hexane (5 ml×2) and the solid was collected to afford compound 52(180 mg). MS (ESI) m/z: 743 [M+H]⁺.

### 2-((2R,4R,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo [4,5-d]pyrimidin-3-yl)tetrahydrofuran-2-yl)ethan-1-ol (Compound 53)

To a stirred solution of compound 52(180 mg, 0.24 mmol) in methanol (10 ml) at 0° C. was added NaBH₄ (90 mg, 2.37 mmol) and the reaction mixture was stirred overnight. After that, the mixture was partitioned between ethyl acetate (40 ml) and brine (40 ml). The aqueous layer was extracted with ethyl acetate (20 ml). The combined organic layer was washed with brine (10 ml×2), dried over anhydrous Na₂SO₄, filtered and concentrated under reduced pressure. The residue was purified by silica gel column chromatography (n-hexane/ethyl acetate, 2:1) to afford compound 53(80 mg). MS (ESI) m/z: 745 [M+H]⁺.

### 2-((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl) ethan-1-ol (Compound 54)

To a solution of compound 53(80 mg, 0.11 mmol) in THF (10 ml) was added TBAF(1 ml, 1M in THF) and the reaction solution was stirred at room temperature overnight. After that, it was concentrated and the residue was partitioned between dichloromethane (30 ml) and water (10 ml). The organic layer was dried over Na₂SO₄, filtered and concentrated. The residue was purified by gel column chromatography (dichloromethane/methanol, 20:1) to afford compound 54(50 mg). MS (ESI) m/z: 507 [M+H]⁺. ¹H-NMR(400 MHZ, CDCl₃): 7.33;(1H,m)), 7.20;(1H,d), 7.10-7.00;(1H,m),5.55-5.65;(1H,m),4.99-4.80;(3H,m),4.18-4.17;(1H,m),3.68-3.61;(1H,m),3.50-3.48;(1H,m),3.10-3.01;(4H,m),2.93-2.85; (1H,m), 2.19-2.17;(1H, t), 1.73-1.68;(2H, m),1.51-1.29;(4H,m), 1.01-0.97; (1H,t).

### Example 11

### The Preparation of 2-(((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5-(hydroxymethyl)tetrahydrofuran-2-yl)methoxy)ethan-1-ol (Compound 56)

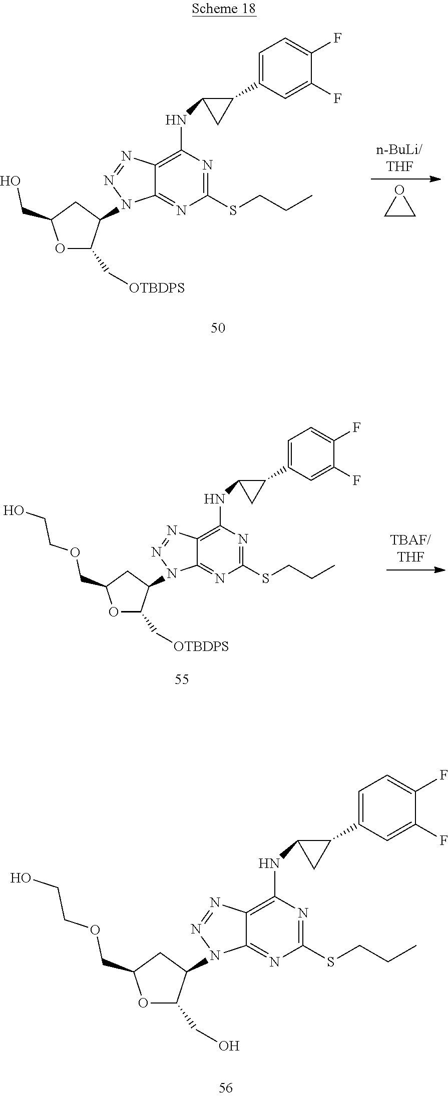

### 2-(((2R,4R,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2-yl)methoxy)ethan-1-ol (Compound 55)

To a solution of compound 50 (200 mg,0.27 mmol) in THF (3ml) under N₂ atmosphere at −78° C. was added n-BuLi (1 ml, 1.6M in THF) and the mixture was stirred at this temperature for 15 min. Ethylene oxide (2.7 ml, 1M in THF) was added and then the mixture was stirred at room temperature overnight. The reaction was quenched with sat.aq. NH₄Cl (10 ml) and partitioned between ethyl acetate (50 ml) was water (20 ml). The organic phase was collected and the aqueous phase was exacted with ethyl acetate (30 ml). The combined organic phase was washed with brine (10 ml×2), dried over anhydrous Na₂SO₄, filtered and concentrated under reduced pressure to afford compound 55(207 mg). MS (ESI) m/z: 775 [M+H]⁺.

### 2-(((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl)methoxy)ethan-1-ol (Compound 56)

To a solution of compound 55(207 mg, 0.27 mmol) in THF(6 ml) was added TBAF (2 ml, 1M in THF) and the reaction solution was stirred at room temperature overnight. After that, it was concentrated and the residue was partitioned between dichloromethane (50 ml) and water(10 ml). The organic layer was dried over Na₂SO₄, filtered and concentrated under reduced pressure. The residue was purified by C₁₈ column chromatography (acetonitrile/water, 20:1) to afford compound 56(50 mg). MS (ESI) m/z: 537 [M+H]⁺. ¹H-NMR (400 MHZ, CDCl₃): 7.10-6.99;(2H,m),6.82-6.73;(1H,m), 4.48-4.37;(1H,m),4.33-4.22;(1H,m),4.09-4.06;(1H,m),4.00-3.86;(2H,m),3.82-3.73;(2H,m),3.56-3.53;(2H,m),3.53-3.40;(2H,m),3.30-2.89;(4H,m),2.80-2.60;(1H,m),2.28-2.18;(1H,m), 1.70-1.60;(2H,m), 1.59-1.43;(2H,m),0.92-0.89;(3H,t).

### Example 12

### The Preparation of 3-((2S,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5-(hydroxymethyl)tetrahydrofuran-2-yl)propanoic Acid (Compound 60)

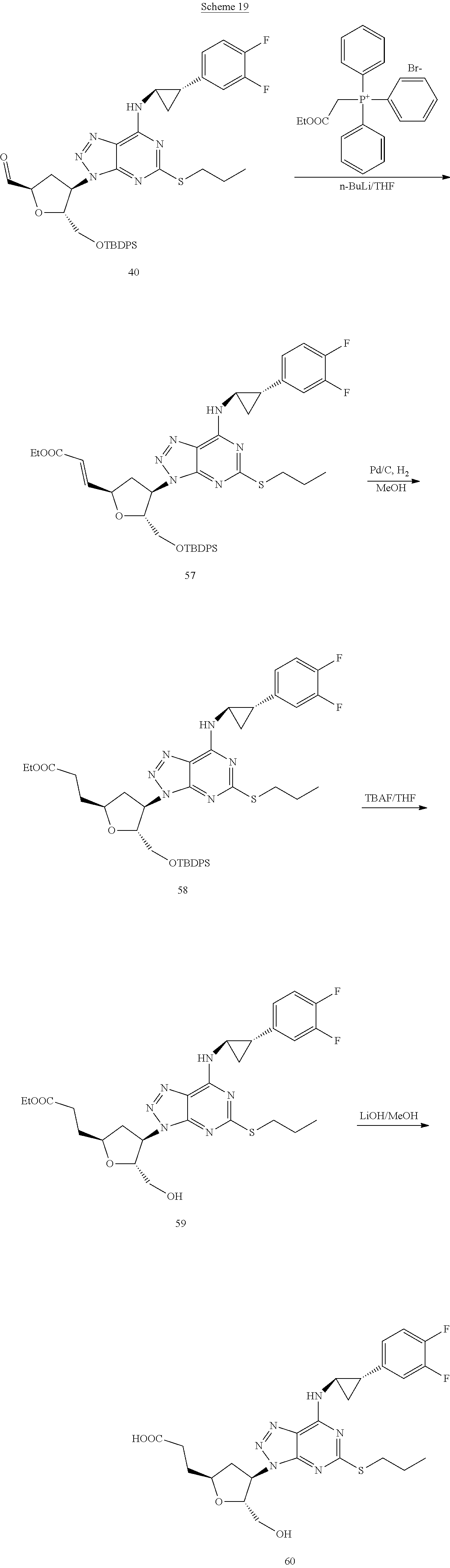

### Ethyl (E)-3-((2R,4R,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2-yl) acrylate (Compound 57)

To a solution of (carbethoxymethyl) triphenylphosphonium bromide (1.71 g,4 mmol) in THF(5 ml) under N₂ atmosphere at −78° C. was dropped n-BuLi (1 ml, 4M in THF) and the resulting mixture was stirred at this temperature for 15 min. A solution of compound 40(200 mg, 0.27 mmol) in THF (1.5 ml) was added and then the reaction mixture was further stirred at room temperature overnight. The reaction was quenched with sat.aq NH₄Cl (5 ml) and partitioned between ethyl acetate (30 ml) and water (30 ml). The organic phase was washed with brine (10 ml×2), dried over anhydrous Na₂SO₄ and concentrated under reduced pressure. The residue was purified by silica gel column chromatography (n-hexane/ethyl acetate, 15:1 to 5:1) to afford compound 57(100 mg). MS (ESI) m/z: 799 [M+H].

### Ethyl 3-((2S,4R,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2-yl)propanoate (Compound 58)

A mixture of compound 57(95 mg, 0.12 mmol) and Pd/C (20 mg) in methanol (10 ml) and ethyl acetate (10 ml) was stirred under H2 atmosphere overnight. After that, the mixture was filtered and concentrated under reduced pressure to afford compound 58 (90 mg). MS (ESI) m/z: 801 [M+H]⁺

Ethyl 3-((2S,4R,5R)-4-(7-(((1R,2S)-2-(3,4-difluorophenyl)cyclopropyl)amino)-5-(propylthio)-3H-[1,2,3]triazolo[4,5-d]pyrimidin-3- yl)-5-(hydroxymethyl)tetrahydrofuran-2-yl)propanoate (Compound 59)

To a solution of compound 58(90 mg, 0.11 mmol) in THF (3 ml) was added TBAF (0.5ml, 1M in THF) and the reaction solution was stirred at room temperature overnight. After that, the solution was concentrated and then partitioned between ethyl acetate (20 ml) and water (20 ml). The organic phase was washed with brine (10 ml), dried over anhydrous Na₂SO₄, filtered and evaporated under reduced pressure. The residue was purified by silica gel column chromatography (n-hexane/ethyl acetate, 5:1 to 1:1) to afford compound 59(53 mg). MS (ESI) m/z: 563 [M+H]⁺

### 3-((2S,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl)propanoic Acid (Compound 60)

To a solution of compound 59 (50 mg, 0.09 mmol) in THF (3 ml) and water (3 ml) was LiOH (10 mg, 0.42 mmol) and the resulting reaction mixture was stirred at room temperature overnight. After that, it was adjusted to PH=4 and partitioned between ethyl acetate (20 ml) and water (20 ml). The aqueous phase was further exacted with ethyl acetate (10 ml×2). The organic phases were combined, dried over anhydrous Na₂SO₄, filtered and concentrated under reduced pressure. The residue was purified by C₁₈ column chromatography (methanol/water, 1:5) to afford compound 60(30 mg). MS (ESI) m/z: 535 [M+H]⁺. ¹H-NMR(400 MHZ, CDCl₃): 7.17-7.00;(3H,m),6.49;(1H,s),5.36-5.34;(1H,q),4.45-4.43;(1H,q), 4.40-4.34;(1H,m), 3.85-3.81;(1H,dd),3.66-3.63;((1H,dd),3.10-3.07;(3H,m),2.77-2.71;(1H,m),2.52-2.48;(1H,m),2.15-2.05;((3H,m), 1.73-1.68;(2H,m), 1.40-1.37;(2H,m), 1.00-0.97;(3H,t).

### Example 13

### The Preparation of 2-((((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5-(hydroxymethyl)tetrahydrofuran-2-yl)methoxy)methyl)benzoic Acid (Compound 63)

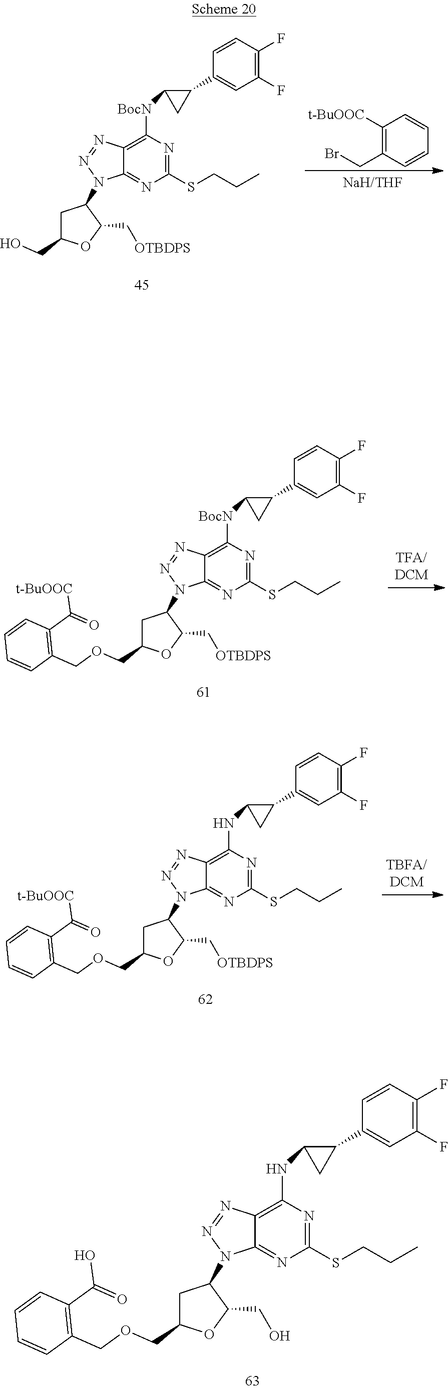

### Tert-butyl 2-((((2R,4R,5R)-4-(7-((tert-butoxycarbonyl)((1R,2S)-2-(3,4-difluorophenyl)cyclopropyl)amino)-5-(propylthio)-3H-[1,2,3]triazolo[4,5-d]pyrimidin-3-yl)-5-(((tert-butyldiphenylsilyl)oxy)methyl)tetrahydrofuran-2-yl)methoxy)methyl)benzoate (Compound 61)

To a solution of compound 45(125 mg, 0.15 mmol) in dry THF(15 m) was added 60% NaH (60 mg,1.5 mmol) in mineral oil at 0° C. under N₂ atmosphere and the resulting mixture was stirred for 30 min. After that, tert-butyl 2-(bromomethyl)benzoate (200 mg, 0.74 mmol) was added and the mixture was allowed to warm to room temperature overnight. The reaction was quenched with sat. aq.NH₄Cl(20 ml) and extracted with ethyl acetate(30 ml×3). The organic phase was washed with brine(30 ml), dried over anhydrous Na₂SO₄, filtered and concentrated under reduce pressure. The residue was further purified by silica gel column (n-hexane/ethyl acetate, 10:1) to afford compound 61(110 mg), MS (ESI) m/z: 1021 [M+H]⁺.

### 2-((((2R,4R,5R)-5-(((tert-butyldiphenylsilyl)oxy)methyl)-4-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2-yl)methoxy)methyl)benzoic Acid (Compound 62)

To a solution of compound 61(110 mg, 0.11 mmol) in dichloromethane (6 ml) was added TFA (2 ml) and the resulting solution was stirred for 3 h. After that, sat.aq. NaHCO₃ was added to adjust to PH=7 and the mixture was exacted with dichloromethane (30 ml×2). The organic phase was washed with brine(30 ml), dried over anhydrous Na₂SO₄, filtered and concentrated under reduced pressure. The residue was purified by silica gel column chromatography (n-hexane/ethyl acetate,6:1) to afford compound 62 (60 mg). MS (ESI) m/z: 865 [M+H]⁺.

### 2-((((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3- yl)-5-(hydroxymethyl)tetrahydrofuran-2-yl)methoxy)methyl)benzoic Acid (Compound 63)

To a solution of compound 62(60 mg, 0.07 mmol) in dry THF (6 ml) was added TBAF (1M in THE, 0.5 ml) and the resulting mixture was stirred at room temperature for 3 h. After that, the reaction mixture was partitioned between ethyl acetate(60 ml) and water(30 ml). The organic phase was washed with brine (30ml), dried over anhydrous Na₂SO₄ and evaporated under reduced pressure. The residue was purified by C18 column chromatography (acetonitrile/H₂O, 9:1˜1:1) to afford compound 63(25 mg). MS (ESI) m/z: 627 [M+H]⁺. ¹H-NMR (400 MHZ, MeOD) 8.03-8.01;(1H,dd),7.44-7.40;(1H,t),7.35-7.31;(1H,t), 7.23-7.18;(1H,d),7.15-6.78;(3H,m),6.22-6.10;(0.5H,m),5.83-5.59;(1H,m),5.50-5.44;(0.5H,m),5.41-5.35;(1H,q),4.53;(1H,s), 4.41-4.32;(1H,s), 3.81-3.78;(3H.m),3.65-3.59;(1H,m),3.18-3.11;(1H,m),2.98-2.79;(2H,m),2.66-2.62;(2H,t),2.46-2.25;(1H,m), 1.72-1.63;(2H,m), 1.43-1.26;(2H,m), 1.03-0.89;(3H,t).

### Example 14

### The Preparation of 2-(((2R,4R,5R)-4-(7-(((1S,2S)-2-(3,4-difluorophenyl)cyclopropyl)amino)-5-(propylthio)-3H-[1,2,3]triazolo[4,5-d]pyrimidin-3-yl)-5-(hydroxymethyl)tetrahydrofuran-2-yl)methoxy)acetic Acid (Compound 65)

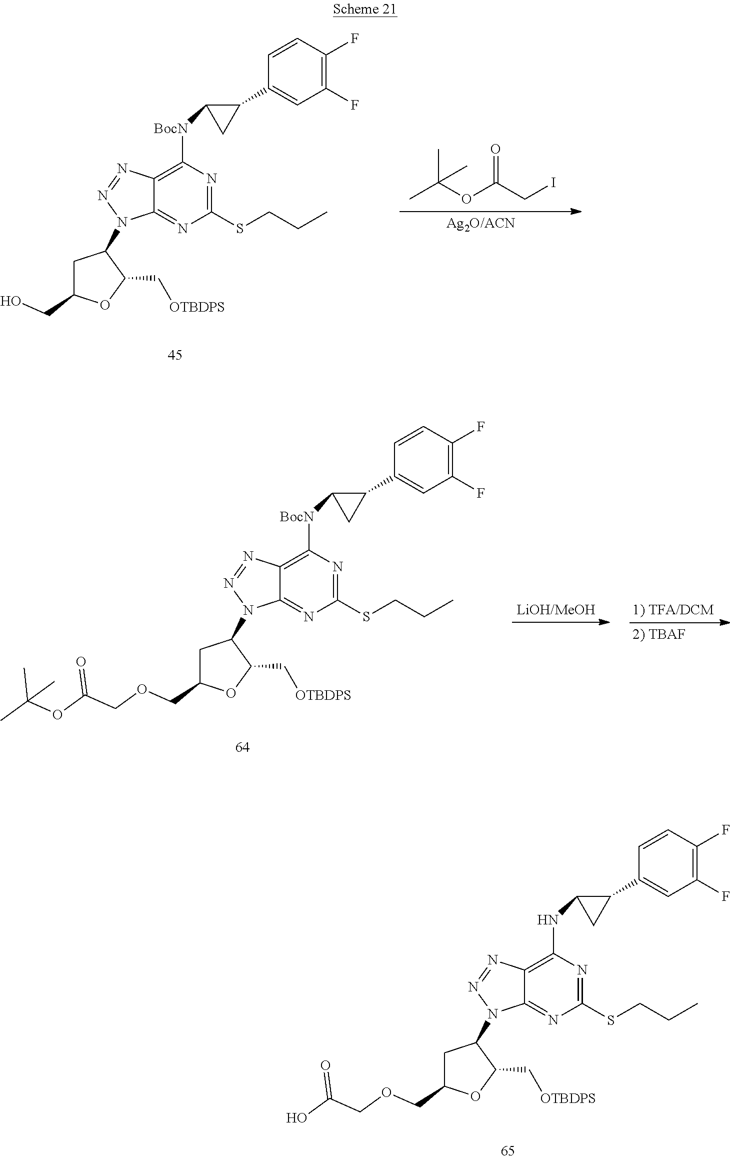

### Tert-butyl 2-(((2R,4R,5R)-4-(7-((tert-butoxycarbonyl)((1R,2S)-2-(3,4-difluorophenyl)cyclopropyl)amino)-5-(propylthio)-3H-[1,2,3]triazolo[4,5-d]pyrimidin-3-yl)-5-(((tert-butyldiphenylsilyl)oxy)methyl)tetrahydrofuran-2-yl)methoxy)acetate (Compound 64)

To a solution of compound 45 (200 mg, 0.24 mmol) in acetonitrile (10 ml) was added ethyl 2-iodoacetate (513 mg, 2.4 mmol) and Ag2O (557 mg, 2.4 mmol) and the resulting mixture was stirred at 60° C. for three days. After that, the mixture was filtered and concentrated under reduced pressure. The residue was purified by silica gel column chromatography (n-hexane/ethyl acetate, 10:1) to afford compound 64(120 mg). MS (ESI) m/z: 945 [M+H]⁺.

### 2-(((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl)methoxy)acetic Acid (Compound 65)

To a solution of compound 64 (120 mg, 0.13 mmol) in dichloromethane (10 ml) was added TFA (3 ml) and the resulting solution was stirred at room temperature overnight. Then it was partition between dichloromethane (50 ml) and sat.aq NaHCO₃ (30 ml). The organic phase was washed with brine (20 ml), dried over anhydrous Na₂SO₄, filtered and concentrated under reduced pressure. The residue was dissolved in THF (5 ml) which followed with addition of TBAF (1 ml, 1M in THF). The mixture was stirred at room temperature overnight and it was partitioned between ethyl acetate (50 ml) and sat.aq. NH₄Cl (30 ml). The organic phase was washed brine (10 ml), dried over anhydrous Na₂SO₄, filtered and evaporated, The residue was purified by C₁₈ column chromatography (acetonitrile/water. 1:10) to afford compound 64(50 mg). MS (ESI) m/z: 551 [M+H]⁺. ¹H-NMR (400 MHZ, DMSO): , 7.36-7.31;(2H,m),7.10-7.03;(1H,m),5.34-5.28;(1H,q),4.44-4.35;(2H,m),3.78-3.72;(1.5H,m),3.61-3.53;(2.5H,m),3.48-3.43;(2H,m),3.12-3.07;(2H,m),2.97-2.85;(2H,m),2.64-2.58;(1H,m),2.15-2.11;(1H,m),1.57-1.48;(3H,m),1.41-1.34;(1H,m), 1.01-0.98;(0.4m,t),0.85-0.81;(2.6H,t)

### Biological Assays

### The Inhibition Study in Human P2Y₁₂ Receptor Transfected CHO Cells

A CHO cell line which stably expresses human P2Y₁₂ receptors was used in the study of the inhibitory effect of the compounds of the disclosure. (The cell line was constructed by Beijing Ion Channel Explorer Co.) The hP2Y12-CHO Cells were cultured in Ham's F-12 medium (Ham's F-12 Medium: +10% FBS +200 μg/ml Hygromycin B) and incubated at 37° C. under 5% CO₂. The poor culture medium was removed, and the cells were washed with PBS. Then 1 mL trypsin solution was added to digest/detach the cells. The culture dish was incubated at 37° C. for 2 minutes. As soon as the cells were detached, 5 mL 37° C. pre-warmed complete medium was added. Cell suspension was transferred into a sterile pipet, the cells aggregates were dissociated by a gentle homogenization. The cell suspension was transferred into a sterile tube, and centrifuged at 1000 rpm for 5 minutes; In order to maintain the physiological activity of cells, the degree of cell fusion was controlled at about 80%.

After cell counting, the cells were cultured in 384-well plate. The test compounds were serially diluted 5-fold from 10000 nM for 10 concentrations and in 3 replicates. Ticagrelor was used as the reference compound and 2-MeSADP as the inducer. After the incubation for 15 min at 37° C., forskolin buffer was added to all wells to induce the production of cAMP. The plate was centrifuged at 1000 rpm for 1 minute, followed by incubation at 37° C. for 30 min. After lysis, the cAMP expression was quantified using PerkinElmer's LANCE® Ultra cAMP Kit method. The inhibitory effect of the test compounds were calculated as percent response to 2-MeSADP in the absence of antagonist and the IC₅₀ was calculated by fitting % Inhibition values and log of compound concentrations to non-linear regression with GraphPad 7.0.

Representative results of some exemplified compounds of the disclosure from the assays are presented in Table 1.

### The Inhibitory Effect on P2Y₁₂ in human P2Y₁₂ Receptor Transfected CHO Cells

Category A: <0.05 μM; Category B: 0.05-0.25 μM; Category C: 0.25-1.25 μM; Category D: >1.25 μM.

TABLE 1

| | Inbibition of P2Y₁₂ |
|---|---|
| Compounds | IC₅₀ (μM) |
| Compound 8 | C |
| Compound 19 | A |
| Compound 27 | A |
| Compound 32 | A |
| Compound 39a | B |
| Compound 39b | B |
| Compound 42 | A |
| Compound 43 | C |
| Compound 49 | C |
| Compound 54 | D |
| Ticagrelor | A |

What is claimed is:

1. A compound of general Formula (I): 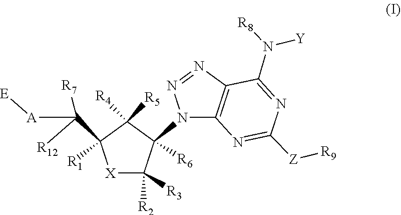
Wherein,
X represents O, S, —S(O)— or —S(O)₂—;
Z represents S, O, NR₁₀, NH or a single bond;
R₁₀ represents C₁₋₆ alkyl;
R₉ represents H, halogen, C₁₋₈ alkyl, C₂₋₈ alkenyl, C₂₋₈ alkynyl, C₁₋₈ alkyl-carbonyl, C₃₋₈ cycloalkyl, aryl, arakyl, 4-6 membered heterocyclyl or 4-6 membered heterocyclylalkyl, wherein each said group may optionally be substituted by one or more substituents independently selected from halogen, hydroxyl, C₁₋₃ alkoxy, C₁₋₃ alkylthio, amino, N-monoalkylamino, N,N-di-alkylamino, cyano and nitro;
R₈ represents hydrogen, C₁₋₆ alkyl, C₁₋₆alkyloxycarbonyl, N-C₁₋₃ alkylcarbamoyl or N,N-diC₁₋₃ alkylcarbamoyl, wherein said C₁₋₆alkyl, N-C₁₋₃ alkylcarbamoyl and N,N-diC₁₋₃ alkylcarbamoyl groups may optionally be substituted by one or more substituents selected from hydroxyl, halogen, C₁₋₃ alkoxy;
Y represents H, C₁₋₈ alkyl, C₁₋₆ alkoxy-C₁₋₆ alkyl, C₁₋₆ alkyl-thio-C₁₋₆ alkyl, C₃₋₈ cycloalkyl, C₃₋₈cycloalkyl-C₁₋₃ alkyl, phenyl-C₁₋₃ alkyl, phenyl-C₃₋₈ cycloalkyl, 4-10membered heterocyclyl, 4-10 membered heterocyclyl-C₁₋₃ alkyl or 4-10membered-heterocyclyl-C₃₋₈cycloalkyl, wherein each said group may optionally be substituted by one or more substituents independently selected from halogen, hydroxyl, cyano, nitro, C₁₋₃ alkyl, C₂₋₆ alkenyl, C₂₋₆ alkynyl, C₁₋₃ alkoxy, C₃₋₆ cycloalkyl, C₁₋₃ alkoxy-C₁₋₃ alkyl, halo-C₁₋₃ alkyl, amino, N-alkylamino, and N,N-dialkylamino;
R₁ represents H, cyano, C₁₋₃ alkyl, halo-C₁₋₃ alkyl, hydroxylmethyl, 2-hydroxyethyl, C₁₋₈ alkylcarboxylmethyl, or benzoylmethyl, wherein the said C₁₋₈ alkylcarboxylmethyl, and benzoylmethyl groups may optionally be substituted by one or more substituents selected from halogen, carboxyl and C₁₋₆ alkoxy-carbonyl;
R₂ and R₃ independently represent hydrogen, C₁₋₃ alkyl, halo-C₁₋₃ alkyl, C₁₋₃alkoxy, hydroxylmethyl, 2-hydroxyethyl, 3-hydroxypropyl, —C O)OH, —CH₂C(O)OH, C₁₋₆ alkoxycarbonyl, C₁₋₆ alkoxycarbonyl methyl or cyano; or R₂ and R₃ together with the carbon to which they are bonded form a C₃₋₅ carbocyclyl, wherein the said carbocyclyl may optionally be substituted by one or more substituents selected from halogen, OH, C₁₋₃ alkyl or C₁₋₃ alkoxy;
R₄ and R₅ independently represent hydrogen, halogen, hydroxyl, C₁₋₃ alkyl, halo-C₁₋₃ alkyl, C₁₋₃ alkoxy, hydroxylmethyl, 2-hydroxyethyl, 3-hydroxypropyl, —C(O)OH, C₁₋₆ alkoxycarbonyl, C₁₋₆ alkoxycarbonylmethyl, cyano, amino, N-alkylamino or N,N-dialkylamino; or R⁴ and R⁵ together with the carbon to which they are bonded form a C₃₋₅ carbocyclyl, wherein the said carbocyclyl may optionally be substituted by one or more substituents with halogen, OH, C₁₋₃ alkyl or C₁₋₃ alkoxy;
R₆ represents hydrogen, cyano, C₁₋₃ alkyl, hydroxylmethyl or 2-hydroxyethyl;
R₇ represents H, F, C₁₋₃ alkyl, hydroxymethyl, 2-hydroxyethyl or HOC(O)CH₂—; or R₇ and R₁₂ together represent an oxo;
A represents a single bond, H, O, S, CH₂, CH₂CH₂, CHF, CF₂ Or C(O);
E represents hydrogen, F, —OH, R₁₁O—, RuOC(O)—, R₁₁C(O)—, R₁₁C(O)O— or a substructure selected from G1, G2 and G3; 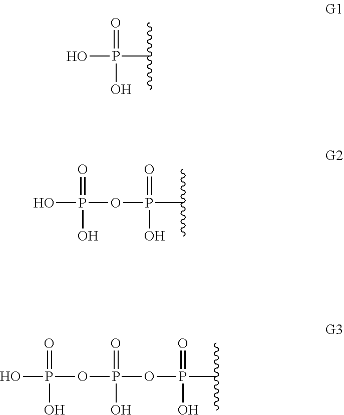 [SMILES: *P(=O)(O)O ; *P(=O)(O)OP(=O)(O)O ; *P(=O)(O)OP(=O)(O)OP(=O)(O)O]
R₁₁ represents hydrogen, C₁₋₈ alkyl, C₃₋₈ alkenyl, C₃₋₈ alkynyl, C₃₋₈ cycloalkyl, benzyl, aryl or heterocyclyl, wherein the said groups may optionally be substituted by one or more substituents selected from halogen, OH, nitro, cyano, C₁₋₃ alkyl, C₁₋₃ alkoxy, —C(O)OH, C₁₋₆ alkoxycarbonyl and C₁₋₆ alkoxycarbonyloxy;
R₁₂ represents hydrogen; or R₁₂ and R₇ together represent an oxo;
with the proviso that Y is not H when R₈ is H;
and with the proviso that R₈ is not H when Y is H;
and with the proviso that R₉ is not H when Z is a single bond;
or a pharmaceutically acceptable salt or prodrug thereof.

2. A compound according to claim 1, wherein
X is O and R₆ is H;
with the proviso that Y is not H when R₈ is H;
and with the proviso that R₈ is not H when Y is H;
and with the proviso that R₉ is not H when Z is a single bond;
or a pharmaceutically acceptable salt or prodrug thereof.

3. A compound according to claim 1, wherein X is O; R₁ is H; R₆ is H; R₁₂ is H; R₈ is H and Z is S.

4. A compound according to claim 1, wherein X is O; R₆ is H; R₁ is H; R₁₂ is H; R₈ is H and Z is S; R₇ is H; R₉ is C₁₋₅ alkyl, C₃₋₈ cycloalkyl or a phenyl group; Y is a cyclopropyl that is substituted with phenyl or heterocyclyl, wherein the said phenyl or heterocyclyl may optionally be substituted by one or more substituents independently selected from halogen, CN, nitro, OH, C₁₋₃ alkyl, C₁₋₃ alkoxy, C₃₋₆ cycloalkyl, amino, N-alkylamino and N,N-dialkylamino.

5. A compound according to claim 1, wherein A is a single bond, H, or O; X is O; R₆ is H; R₁ is H; R₁₂ is H; R₈ is H and Z is S; R₇ is H; R₉ is C₁₋₅ alkyl, C₃₋₈ cycloalkyl or a phenyl group; Y is a cyclopropyl that is substituted with phenyl or heterocyclyl, wherein the said phenyl or heterocyclyl may optionally be substituted by one or more substituents independently selected from halogen, CN, nitro, OH, C₁₋₃ alkyl, C₁₋₃ alkoxy, C₃₋₆ cycloalkyl, amino, N-alkylamino and N,N-dialkylamino.

6. A compound according to claim 1, wherein R₁₂ is H; R₄ and R₅ are independently selected from H, fluorine and hydroxyl.

7. A compound according to claim 1, wherein R₁ is H; R₆ is H, R₁₂ is H; R₈ is H, Z is S; R₃ is H, A is O or a single bond; E is H, R₁₁O—or R₁₁C(O)—; R₁₁ is C₁₋₈ alkyl, benzyl, heterocyclyl or phenyl, wherein the said C₁₋₈ alkyl, benzyl, phenyl or heterocyclyl groups may optionally be substituted by one or more substituents selected from halogen, OH, C₁₋₃ alkyl, C₁₋₃ alkoxy, carboxyl, C₁₋₆ alkoxycarbonyl and C₁₋₆ alkoxycarbonyloxy.

8. A compound according to claim 1, wherein R₁₂is H; R₃ is H and R₂ is hydroxymethyl.

9. A compound according to claim 1, wherein Y is a cyclopropyl that is substituted with a phenyl, wherein the said phenyl is substituted by 1 to 4 fluorine atoms.

10. A compound according to claim 1, wherein selected from:
((2R,3R,4R,5S)-3-(7-(((1R,2S)-2-(3,4-difluorophenyl)cyclopropyl)amino)-5-(propylthio)-3H-[1,2,3]triazolo[4,5-d]pyrimidin-3-yl)- 4-hydroxytetrahydrofuran-2,5-diyl) dimethanol;
((2R,3R,5R)-3-(7-(((1R,2S)-2-(3,4-difluorophenyl)cyclopropyl)amino)-5-(propylthio)-3H-[1,2,3]triazolo[4,5-d]pyrimidin-3- yl)tetrahydrofuran-2,5-diyl)dimethanol;
((2R,4R)-4-(7-(((1R,2S)-2-(3,4-difluorophenyl)cyclopropyl)amino)-5-(propylthio)-3H-[1,2,3]triazolo[4,5-d]pyrimidin-3-yl)tetrahydrofuran-2-yl)methanol;
(2S,3R,4S)-4-(7-(((1R,2S)-2-(3,4-difluorophenyl)cyclopropyl)amino)-5-(propylthio)-3H-[1,2,3]triazolo[4,5-d]pyrimidin-3-yl)-2- (hydroxymethyl)tetrahydrofuran-3-ol;
((2R,3S,4S,5S)-3-(7-(((1R,2S)-2-(3,4-difluorophenyl)cyclopropyl)amino)-5-(propylthio)-3H-[1,2,3]triazolo[4,5-d]pyrimidin-3-yl)- 4-fluorotetrahydrofuran-2,5-diyl)dimethanol;
((2R,3S,4R,5S)-3-(7-(((1R,2S)-2-(3,4-difluorophenyl)cyclopropyl)amino)-5-(propylthio)-3H-[1,2,3]triazolo[4,5-d]pyrimidin-3-yl)- 4-fluorotetrahydrofuran-2,5-diyl)dimethanol;
(2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5- (hydroxymethyl)tetrahydrofuran-2-carboxylic acid;
(2R,4R,5R)-4-(7-(((1R,2S)-2-(3,4-difluorophenyl)cyclopropyl)amino)-5-(propylsulfinyl)-3H-[1,2,3]triazolo[4,5-d]pyrimidin-3-yl)-5-(hydroxymethyl)tetrahydrofuran-2-carboxylic acid;
((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)-5- (methoxymethyl)tetrahydrofuran-2-yl)methanol;
2-((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl)ethan-1-ol;
2-(((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl)methoxy)ethan-1-ol;
3-((2S,4R,5R)-4-(7-(((1R,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl)propanoic acid;
2-((((2R,4R,5R)-4-(7-(((1R,2S)-2-(3,4-difluorophenyl)cyclopropyl)amino)-5-(propylthio)-3H-[1,2,3]triazolo[4,5-d]pyrimidin-3- yl)-5-(hydroxymethyl)tetrahydrofuran-2-yl) methoxy)methyl)benzoic acid;
2-(((2R,4R,5R)-4-(7-(((1S,2S)-2-(3,4-difluorophenyl)cyclopropyl)amino)-5-(propylthio)-3H-[1,2,3]triazolo[4,5-d]pyrimidin-3-yl)- 5-(hydroxymethyl)tetrahydrofuran-2-yl)methoxy)acetic acid;
or a pharmaceutically acceptable salt or prodrug thereof.

11. A pharmaceutical composition for the treatment, amelioration or prevention of a platelet-mediated thrombotic diseases, disorders or conditions including myocardial infarction, thrombotic stroke, transient ischemic attacks, peripheral vascular disease, angina and unstable angina in a host comprising an effective amount of a compound according to claim 1.

12. The pharmaceutical composition according to claim 11, which further comprises one or more additional antiplatlet, anticoagulant, antifibrinolytic agents or other therapeutic agents having cardiovascular effect.

13. A compound according to claim 1 ,for use in therapy.

14. A compound according to claim 1, for use in the treatment, amelioration or prevention of a platelet-mediated thrombotic diseases, disorders or conditions including myocardial infarction, thrombotic stroke, transient ischaemic attacks, peripheral vascular disease, angina and unstable angina.

15. A compound according to claim 14, which use further comprises one or more additional antiplatlet, anticoagulant, antifibrinolytic agents or other therapeutic agents having cardiovascular effect.

16. (canceled)

17. (canceled)

18. A method for the treatment or prevention of a platelet-mediated thrombotic diseases, disorders or conditions including myocardial infarction, thrombotic stroke, transient ischaemic attacks, peripheral vascular disease, angina and unstable angina in a subject in need thereof, comprising administering a therapeutically effective amount of a compound according to claim 1.

19. The method according to claim 18, which further comprises one or more additional antiplatlet, anticoagulant, antifibrinolytic agents or other therapeutic agents having cardiovascular effect.
